SEQUENCE LISTING (16 sequences)

SEQ ID NO: 1 - DNA, 20 bp, Artificial Sequence
  primer
cactcttccagccttccttc

SEQ ID NO: 2 - DNA, 20 bp, Artificial Sequence
  primer
gtacaggtctttgcggatgt

SEQ ID NO: 3 - DNA, 20 bp, Artificial Sequence
  primer
gccgctagaggtgaaattct

SEQ ID NO: 4 - DNA, 20 bp, Artificial Sequence
  primer
tcggaactacgacggtatct

SEQ ID NO: 5 - DNA, 28 bp, Artificial Sequence
  primer
gcaaaataccaccttggcgcctacacgg

SEQ ID NO: 6 - DNA, 24 bp, artificial sequence
  primer
gtgacacgggtgtagatgccaggc

SEQ ID NO: 7 - DNA, 20 bp, artificial sequence
  primer
cctgctgctctttctcttgt

SEQ ID NO: 8 - DNA, 22 bp, artificial sequence
  primer
gctccaagggtgactgttatag

SEQ ID NO: 9 - DNA, 20 bp, artificial sequence
  primer
tctatgctctcgcacctttc

SEQ ID NO: 10 - DNA, 20 bp, artificial sequence
  primer
gactcatgggcttgggaata

SEQ ID NO: 11 - DNA, 22 bp, artificial sequence
  primer
ccttgaaccctccatggaatag

SEQ ID NO: 12 - DNA, 22 bp, artificial sequence
  primer
cactttgcccgtacagtagtag

SEQ ID NO: 13 - DNA, 1953 bp, Homo sapiens
acagaacccagaaaaacaactcattcgctttcatttcctcactgactataaaagaatagagaaggaagggcttcagtgaccggctgcctggctgacttacagcagtcagactctgacaggatcatggctatgatggaggtccaggggggacccagcctgggacagacctgcgtgctgatcgtgatcttcacagtgctcctgcagtctctctgtgtggctgtaacttacgtgtactttaccaacgagctgaagcagatgcaggacaagtactccaaaagtggcattgcttgtttcttaaaagaagatgacagttattgggaccccaatgacgaagagagtatgaacagcccctgctggcaagtcaagtggcaactccgtcagctcgttagaaagatgattttgagaacctctgaggaaaccatttctacagttcaagaaaagcaacaaaatatttctcccctagtgagagaaagaggtcctcagagagtagcagctcacataactgggaccagaggaagaagcaacacattgtcttctccaaactccaagaatgaaaaggctctgggccgcaaaataaactcctgggaatcatcaaggagtgggcattcattcctgagcaacttgcacttgaggaatggtgaactggtcatccatgaaaaagggttttactacatctattcccaaacatactttcgatttcaggaggaaataaaagaaaacacaaagaacgacaaacaaatggtccaatatatttacaaatacacaagttatcctgaccctatattgttgatgaaaagtgctagaaatagttgttggtctaaagatgcagaatatggactctattccatctatcaagggggaatatttgagcttaaggaaaatgacagaatttttgtttctgtaacaaatgagcacttgatagacatggaccatgaagccagtttttttggggcctttttagttggctaactgacctggaaagaaaaagcaataacctcaaagtgactattcagttttcaggatgatacactatgaagatgtttcaaaaaatctgaccaaaacaaacaaacagaaaacagaaaacaaaaaaacctctatgcaatctgagtagagcagccacaaccaaaaaattctacaacacacactgttctgaaagtgactcacttatcccaagagaatgaaattgctgaaagatctttcaggactctacctcatatcagtttgctagcagaaatctagaagactgtcagcttccaaacattaatgcaatggttaacatcttctgtctttataatctactccttgtaaagactgtagaagaaagagcaacaatccatctctcaagtagtgtatcacagtagtagcctccaggtttccttaagggacaacatccttaagtcaaaagagagaagaggcaccactaaaagatcgcagtttgcctggtgcagtggctcacacctgtaatcccaacattttgggaacccaaggtgggtagatcacgagatcaagagatcaagaccatagtgaccaacatagtgaaaccccatctctactgaaagtacaaaaattagctgggtgtgttggcacatgcctgtagtcccagctacttgagaggctgaggcaagagaattgtttgaacccgggaggcagaggttgcagtgtggtgagatcatgccactacactccagcctggcgacagagcgagacttggtttcaaaaaaaaaaaaaaaaaaaacttcagtaagtacgtgttatttttttcaataaaattctattacagtatgtcatgtttgctgtagtgctcatatttattgttgtttttgttttagtactcacttgtttcataatatcaagattactaaaaatgggggaaaagacttctaatctttttttcataatatctttgacacatattacagaagaaataaatttcttacttttaatttaatatga

SEQ ID NO: 14 - DNA, 4018 bp, Homo sapiens
caggcagcgctgcgtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaa

SEQ ID NO: 15 - DNA, 391 bp, Macaca mulatta polyomavirus 1
tttatgtttcaggttcagggggaggtggttttttaaagcaagtaaaacctctacagatgtgatatggctgatcatgaacagactgtgaggactgtgagggctgaggggcctgaaatgagccttgggactgtgaatcaatgcctgtttcatgccctgagtcttccatgttcttctccccaccatcttcatttttatcagcattttcctcatcatcatcactgtttcttagccaatttaaaactccagttcccatagccacattaaacttcattttttgatacactgacaaactaaactctttgtccaatctctctttccactccacaattctgccctgaatactttgagcaaactcagccacaggtctgtaccaaattaacataagaagcaa

SEQ ID NO: 16 - DNA, 2123 bp, homo sapiens
tcccagagtcaacagcgagcgagcagccggagcggggaagcagaagccagagaggggaagaatacggcgccccctctctccctcccctcccccttctactttagcctttctgcgcacttcgcttccaagtctccgcgcagccaggagccgctgttgcctcccagcccctgctagctgccccccgagccgagcgcagcgagcgccgccgcccgggccccccggtggggccagggccagcatggagcacctgggtccgcaccacctccacccgggtcacgcagagcccattagcttcggcatcgaccagatcctcaacagcccggaccagggtggctgcatgggacccgcctcgcgcctccaggacggagaatacggccttggctgcttggtcggaggcgcctacacttacggcggcgggggctccgcggccgcgacgggggctggaggagcgggggcctatggtactggaggtcccggcggccccggaggcccggcaggcggcggcggcgcctgcagcatgggtcctctgaccggctcctacaacgtgaacatggccttggcaggcggccccggtcctggcggcggcggcggcagcagcggcggtgccggggcactcagcgctgcgggggtaatccgggtgccggcacacaggccgctcgccggagccgtggcccacccccagcccctggccaccggcttgcccaccgtgccctctgtgcctgccatgccgggcgtcaacaacctcactggcctcaccttcccctggatggagagtaaccgcagatacacaaaggacaggttcacaggtcacccctatcagaaccggacgccccccaagaagaagaagccgcgcacgtccttcacacgcctgcagatctgcgagctggagaagcgcttccaccgccagaagtacctggcctcggccgagcgcgccgccctggccaaggcgctcaaaatgaccgatgcgcaggtcaaaacctggttccagaaccggcggacaaagtggagacggcagactgcggaggaacgggaggccgagaggcagcaagcgaaccgcatcctcctgcagttgcagcaggaggccttccagaagagcctggcacagccgctgcccgctgaccctctgtgcgtgcacaactcgtcgctcttcgccctgcagaatctgcagccgtggtctgacgactcgaccaaaatcactagcgtcacgtcggtggcgtcggcctgcgagtgagcctgcccattctgccctgtgggaccccaggcccactcaggggtcactgaggcctgagacccaggactcctccccaccctcctggcctcagactgcacccaggaggggaacactgccctcgcacggccccgaagggcccccacatttgtgccgacactgttctcccttcggtggaagagctcaagggacaaggacacgcgcccccctcccagaggcgtcccgcacctgtctgaactgttaagaaatctgtttttgtttatttcattttattttaatttttaacgtgggattcagagaaaggcaagggaggtaagggaggaggagcttctggggtccccagggctgtcatctgaatttgccctgggaaaccccttctctgtgacccacttctcatcacacacatggaaacccataggcccacacacaggtggtgtcactgtccctcctggtgtcaccccagagccacacatgggcatctatgggagagtgtcaaccagacagagggtcacagtgtttacactttggaccttacgatcaggcacaggtcaggggtgacacagactcatcctgaacagcatggcactccctccagcacaaacacaaggtcatggccacactgtgacacactacaccacacacaacagccaacagctacaacagcctcacttggtctgccaggcccccaccacacatcccagcccaatccaggtacgcacagacaggttttcacataaatgcagcccatttctccagaacccatttgaggggtgggggggtgttaatttatgcacttataaggtgttttctgtgtaaccattttataaagtgcttgtgtaatttatgtgaaaaaaataaataaaagcctccggatccggaaaaaaaaaaaaaaaaa

(19) United States
(12) Patent Application Publication
(10) Pub. No.: US 2022/0257648 A1
(43) Pub. Date: Aug. 18, 2022

(54) Therapeutic Human Mast Cells, Compositions, and Methods of Treating a Tumor Comprising Administering Autologous Human Mast Cells from Adipose Tissue

(71) Applicant: University of North Carolina at Greensboro, Greensboro, NC (US)

(72) Inventors: Christopher Kepley, Ringgold, VA (US); Jesse Plotkin, Weaverville, NC (US)

(21) Appl. No.: 16/622,829

(22) PCT Filed: Jun. 29, 2018
(86) PCT No.: PCT/US2018/040183

§ 371 Date: Dec. 13, 2019

Related U.S. Application Data
(60) Provisional application No. 62/527,566, filed on Jun. 30, 2017.

(51) Int. Cl. A61K 35/15; C12N 5/0787; A61P 35/00

(52) U.S. Cl. CPC A61K 35/15; C12N 5/0642; C12N 2501/195; C12N 2506/1384; A61P 35/00

(57) ABSTRACT

This disclosure is directed to mast cells obtained from adipose derived stem cells and sensitized with immunoglobulin-E targeted to a cancer antigen; compositions comprising mast cells obtained from adipose derived stem cells and sensitized with immunoglobulin-E targeted to a cancer antigen; and methods of treating a tumor in a subject, comprising administering to a subject a therapeutically effective amount of mast cells obtained from adipose derived stem cells, wherein the mast cells are autologous to the subject and sensitized with immunoglobulin-E targeted to a cancer antigen.

### 1. CROSS REFERENCE TO RELATED APPLICATIONS

This application is a 35 U.S.C. § 371 national phase application of PCT Application No. PCT/US2018/040183 filed on Jun. 29, 2018, which claims priority to U.S. Provisional Patent Application No. 62/527,566 filed on Jun. 30, 2017. The contents of each are hereby incorporated by reference in their entirety.

### 2. FIELD

The invention relates generally to mast cells, immunology, and immunotherapy.

### 3. SEQUENCE LISTING

The instant application contains a Sequence Listing which has been submitted electronically in ASCII text file format and is hereby incorporated by reference in its entirety. Said ASCII copy, created August 11, 2021, is named “961-6_sequence_listing_ST25” and is 14 kb in size.

### 4. BACKGROUND

Human mast cells (MC) are unique in their ability to pre-store and release potentially beneficial anti-cancer mediators, e.g. tumor necrosis factor alpha (TNF-α) and granulocyte-macrophage colony-stimulating factor (GM-CSF) [139, 140]. Human MC have pre-formed and releasable (through FcϵRI) TNF-α within their granules [51, 162, 163]. TNF-α is an anti-cancer agent shown to suppress tumor cell proliferation, induce tumor regression, and used as an adjuvant that enhances the anti-cancer effect of chemotherapeutic agents [164-166]. Human MC also releases GM-CSF upon FcϵRI stimulation [22, 117]. In fact, human MC release copious amounts (2,500-4,000 pg/ml from 10⁵ cells) of GM-CSF upon FcϵRI stimulation [22, 117]. GM-CSF is also being investigated as an anti-breast cancer therapeutic, including as a co-addition for immunotherapies [139, 167-170]. MC also store and release several other potential anti-tumor mediators including ROS, PGD2, IL-9, and heparin [51, 133]. In one study cord blood-derived MC and eosinophils, sensitized with anti-CD20 IgE, were shown to kill CD20-positive cancer cells [101]. The role of mast cells (MC) has been extensively investigated in cancer [61, 91, 93-95, 133-136, 138, 159-161]; yet there remains a need for novel approaches to target autologous MC to tumor antigens.

### 5. SUMMARY

The presently disclosed subject matter provides human mast cells obtained from adipose derived stem cells and sensitized with an immunoglobulin-E targeted to a cancer antigen; a composition comprising human mast cells obtained from adipose derived stem cells and sensitized with an immunoglobulin-E targeted to a cancer antigen; a method of treating a tumor in a human, comprising administering to a human a therapeutically effective amount of human mast cells obtained from adipose derived stem cells, wherein the mast cells are autologous to the human and sensitized with an immunoglobulin-E targeted to a cancer antigen; and a method of preparing human mast cells in vitro comprising the steps of obtaining human mast cells from adipose derived stem cells, and sensitizing the human mast cells with an immunoglobulin-E targeted to a cancer antigen.

In another embodiment of the method of treatment, the human mast cells are administered in a pharmaceutically acceptable carrier. In another embodiment, the administering step is subcutaneous administration, intraperitoneal administration, intratumoral administration, or intravenous administration. In another embodiment, the cancer antigen is an epithelial cancer antigen, a prostate specific cancer antigen, a prostate specific membrane antigen, a bladder cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, or a colorectal cancer antigen. In another embodiment, the epithelial cancer antigen is a breast cancer antigen, gastrointestinal cancer antigen, or a lung cancer antigen. In another embodiment of the method of treatment, cancer cells are killed, tumor angiogenesis is inhibited, tumor metastasis is inhibited, tumor growth is decreased, or tumor growth is inhibited.

### 6. BRIEF DESCRIPTION OF THE FIGURES

FIG. 1A are light microscopy image comparisons of toluidine-blue stained adipose-derived mast cell (top) and normal skin derived mast cells (middle), and a phase contrast image of adipose derived mast cell culture showing characteristic morphology (bottom).

FIG. 1B are images of gene expression in adipose-derived mast cells for four mast cell genes: Tryptase, Chymase, c-kit and FcϵR1α.

FIG. 1C is immunohistochemistry of adipose derived mast cells demonstrating expression of mast cell-specific tryptase and chymase (top) compared to negative controls (bottom).

FIG. 2A. is a bar graph of beta-hexasaminidase release from adipose derived mast cells compared to skin-derived mast cells, and FIG. 2B is a bar graph of GMC-SF production from adipose derived mast cells compared to skin-derived mast cells.

FIG. 3A is a bar graph of percent degranulation from adipose derived mast cells compared to skin-derived mast cells, and FIG. 3B is a bar graph of cytokine release from adipose derived mast cells compared to skin-derived mast cells. Human skin MC (black box) or ADMC (grey box; 10⁶) were challenged with or without (spontaneous) 1 μg/ml anti-FcϵRI antibodies, IgE anti-NP+IgE+antigen (IgE-Ag), or optimal concentrations of the non-IgE dependent secretagogue poly-1-lysine and degranulation (A) or GM-CSF production (B) assessed in the supernatants. Error bars represent ±SD. *p<0.05 comparing skin MC vs. ADMC release. Figure is representative of cells derived from two different donors.

FIG. 4 is a graph depicting surface expression of MC-specific markers assessed by flow cytometry. ADMC were incubated with mouse anti-c-Kit/CD117 (dashed line), FcϵR1α chain (solid line), or isotype control mouse IgG (grey) for 2 hours on ice, washed, and FITC-labelled anti-mouse F(ab)₂ added for one hour.

FIG. 5 are images of time lapse, confocal microscopy of ADMC binding to breast cancer cells. ADMC (10⁵-10⁶) were sensitized with 1 μg/ml of trastuzumab IgE followed by MitoTracker™ Green. The MitoTracker™ Green-loaded ADMC were added to adherent SK-BR-3 (10⁵-10⁶) that had been pre-stained with MitoTracker™ Red and time lapse video taken over six hours. The white circular boundaries and arrows represent starting point and tracking of ADMC (green) at time 0 to SK-BR-3 (red) binding over the six hours. 20X magnification was used to capture the cellular tracking (start and stop).

FIGS. 6A-D are bar graphs related to breast cancer cell-induced ADMC mediator release. ADMC were sensitized with 1 μg/ml anti-HER/neu IgE (clone C6MH3-B1 IgE or trastuzumab IgE), washed, and incubated with SK-BR-3 cells and degranulation (FIG. 6A) or cytokine release (FIG. 6B) assessed. Data are from a single experiment performed on cells derived from three separate donors. Error bars represent ±SD. *p<0.05 compared with non-IgE (spontaneous) release. All values in panel B were significant.

FIG. 6C shows ECD^{HER2} does not induce ADMC mediator release. ADMC were sensitized with 1 μg/ml anti-HER/neu IgE as in (A), washed, and incubated with ECD^{HER2} and mediator release assessed. As a control, optimal concentrations of anti-FcϵRI antibodies were run in parallel. N=2, in triplicate. Error bars represent ±SD.

FIG. 6D shows sera from HER2/neu positive breast cancer patients does not induce ADMC degranulation. Heat inactivated serums from two separate HER2/neu positive breast cancer patients (P1 and P2; see table in Section I.F. infra) or normal control serum (Ctrl) was used to challenge anti-HER/neu IgE (C6MH3-B1 IgE or trastuzumab IgE) sensitized ADMC and β-hexosaminidase release measured as described. Background levels of β-hexosaminidase naturally found in the serums was subtracted from values. Experiment is representative of two separate ADMC preparations each done in duplicate. Error bars represent ±SD.

FIGS. 7A-E are images and graphs related to ADMC killing of human breast cancer cells. FIG. 7A, CellTracker™-red labelled ADMC (7.5×10⁴; shown here as purple cells) were sensitized with 1 μg/ml anti-HER2/neu IgE (clone trastuzumab), washed, and incubated with MitoTracker™-green-stained SK-BR-3 (10⁵) in culture medium containing PI and images taken before (A, left) and after (A; right) 96 hours. The call out box shows increased PI and ADMC degranulation over time. The increased number of red cells indicates breast cancer cell death as indicated by the PI (red) bound to ADMC (purple; Mag 20×). In FIG. 7B, psIgE was substituted for HER2/neu which resulted in no significant SK-BR-3 cell death.

FIG. 7C are bar graphs quantifying overall PI fluorescence before and after incubation. The * indicates significant (p=0.0003) SK-BR-3 cell death at Day 4 compared to Day 0.

FIGS. 7D and E are images depicting ADMC-induced breast cancer cell apoptosis. Anti-HER2/neu IgE-sensitized ADMC (7.5×10⁴) were incubated with SK-BR-3 (1×10⁵) for 72 hours, cytospins made, fixed, and incubated with Alexa Fluor™ 488 labelled, mouse anti-human tryptase (FIGS. 7D and E; green) along with Alexa Fluor™ 647 labelled, mouse anti-human caspase 3 (FIG. 7D; red) or Alexa Fluor™ 647 labelled, isotype control IgG for caspase 3 antibody (FIG. 7E). Two representative panels are shown.

FIGS. 8A-E are images and bar graphs related to mediators from FcϵRI-challenged ADMC induce human breast cancer cell killing. ADMC (1.3×10⁶) were challenged with optimal concentrations of anti-FcϵRI stimuli (70% release) for 24 hours and supernatants (no cells) from these ADMC (FIG. 8A) or media from non-FcϵRI challenged ADMC (FIG. 8B) incubated with the MitoTracker™ green-stained SK-BR-3 (10⁵) in culture medium containing optimal concentrations of PI and images taken before (FIGS. 8A, 8B-left) and after (FIGS. 8A, 8B-right) 96 hours.

FIG. 8C are bar graphs quantifying overall PI fluorescence before and after incubation. The increased number of red cells indicates breast cancer cell death as indicated by the PI (red) and quantified in (FIG. 8C) showing overall PI fluorescence before and after incubation. Graph in (FIG. 8C) represents average PI intensity from two separate experiments (±SD; * indicates p=0.0008).

FIGS. 8D and 8E are images depicting mediators from FcϵRI-challenged ADMC induce human breast cancer cell apoptosis killing ADMC. In FIGS. 8D and 8E the same media from anti-FIERI challenged ADMC were incubated with SK-BR-3 (10⁵) for 72 hours, cytospins prepared, fixed, and incubated with Alexa Fluor 647 labelled, anti-human caspase 3 (FIG. 8D; left, right, red) or Alexa Fluor 647 labelled, isotype control antibody for caspase 3 (FIG. 8E; left, right). Two representative panels are shown.

FIG. 9 is a graphic of autologous mast cell cancer immunotherapy (AMCIT).

### 7. BRIEF DESCRIPTION OF THE SEQUENCE LISTING

SEQ ID NO: 1 represents the beta actin Forward primer.

SEQ ID NO: 2 represents the beta actin Reverse primer.

SEQ ID NO: 3 represents the 18s Forward primer.

SEQ ID NO: 4 represents the 18s Reverse primer.

SEQ ID NO: 5 represents the Tryptase Forward primer.

SEQ ID NO: 6 represents the Tryptase Reverse primer.

SEQ ID NO: 7 represents the Chymase Forward primer.

SEQ ID NO: 84 represents the Chymase Reverse primer.

SEQ ID NO: 9 represents the c-KIT Forward primer.

SEQ ID NO: 10 represents the c-KIT Reverse primer.

SEQ ID NO: 11 represents the FcϵR1a Forward primer.

SEQ ID NO: 12 represents the FcϵR1a Reverse primer.

SEQ ID NO: 13 represents the TRAIL sequence (NM_003810).

SEQ ID NO: 14 represents the hTERT sequence (NM_198253).

SEQ ID NO: 15 represents the SV40 Large T Antigen sequence (M99347).

SEQ ID NO: 16 represents the TLX1 sequence (NM_005521).

### 8. DETAILED DESCRIPTION OF REPRESENTATIVE EMBODIMENTS

The presently disclosed subject matter now will be described more fully hereinafter. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the descriptions and the associated figures. Therefore, it is to be understood that the presently disclosed subject matter is not to be limited to the specific embodiments disclosed and that modifications and other embodiments are intended to be included within the scope of the appended claims.

4.A. Definitions

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety. Numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

As used herein, the term “culture” is used to denote the maintenance or cultivation of cells in vitro including the culture of single cells. Cultures can be cell, tissue, or organ cultures, depending upon the extent of organization.

As used herein, the term “cell line” is used to refer to cells which have arisen from a primary culture and capable of successful subculture.

As used herein, the term “isolated” means removal from its native environment, and can include removal from its immediate native environment.

In the context of the present invention by “purified” or “pure” cells it is meant a population of cells that comprises at least 80%, and more preferably at least 90%, human mast cells. In some embodiments, the human mast cells are at least 95% pure. In some embodiments, the cell human mast cells are at least 99% pure.

As used herein, the term “differentiated” refers to a state of cells in which the cultured cells maintain all, or a substantial amount of, their specialized structure and function typical of the cell type in vivo. Partially differentiated cells maintain less than a substantial amount of their full complement of specialized structure and/or function.

As used herein, the terms “expression” or “gene expression” refer to transcription of a gene into an RNA product, and optionally to translation into one or more polypeptide sequences. The term “transcription” refers to the process of copying a DNA sequence of a gene into an RNA product, generally conducted by a DNA-directed RNA polymerase using DNA as a template.

As used herein, the term “nucleic acid” refers to a polymer of ribonucleic acids or deoxyribonucleic acids, including RNA, mRNA, rRNA, tRNA, small nuclear RNAs, cDNA, DNA, PNA, RNA/DNA copolymers, or analogues thereof. Nucleic acid may be obtained from a cellular extract, genomic or extragenomic DNA, viral RNA or DNA, or artificially/chemically synthesized molecules.

As used herein, the term “RNA” refers to a polymer of ribonucleic acids, including RNA, mRNA, rRNA, tRNA, and small nuclear RNAs, as well as to RNAs that comprise ribonucleotide analogues to natural ribonucleic acid residues, such as 2-O-methylated residues.

As used herein, the term “phenotype” refers to all the observable characteristics of a cell (or organism); its shape (morphology); interactions with other cells and the non-cellular environment (e.g., extracellular matrix); proteins that appear on the cell surface (surface markers); and the cell's behavior (e.g., secretion, contraction, synaptic transmission).

As used herein, the terms “administer”, “apply”, “treat”, “transplant”, “implant”, “deliver”, and grammatical variations thereof, are used interchangeably to provide mast cells of the invention to a mammal, or, depending upon the context, contacting a potential modulator of biological activity (candidate agent) with a mast cell of the invention.

As used in this specification and the appended claims, the singular forms “a”, “an”, and “the” include plural reference unless the context clearly dictates otherwise. Thus, for example, a reference to “a mast cell” or “a human mast cell” includes one or more of such cells.

4.B. Mast Cells

Mast cells are myeloid hematopoietic cells, or immune cells, that are derived from hematopoietic stem cells in the bone marrow [1]. Mast cell progenitors leave the bone marrow and differentiate into different mast cell subtypes corresponding to the tissue compartments of end organ tissue. Mast cells are found in virtually every tissue in the human body, including mucosal and connective tissues throughout the body. However, isolation of mast cells is difficult due to the relatively low abundance and distribution of mast cells in a wide variety of tissues.

It has been demonstrated that mature mast cells can proliferate from progenitors found in human skin [2]. However, obtaining autologous mast cells from patients has not yet been practical given the intrusiveness of obtaining and proliferating large enough numbers for patient use (e.g. from skin). The skin-derived MC (MCTC) proliferate in culture and retain the MCTC protease phenotype of MC that normally reside in the dermis [3]. After 4 to 8 weeks of culture these cells degranulate in response to IgE stimulus, substance P and compound 48/80, characteristics of skin-derived MC that persist outside of the cutaneous microenvironment. Lung-derived mast cells (predominately MCT) do not proliferate in culture and must be used within 1 to 2 weeks after purification.

Another problem faced in isolating tissue mast cells is costs associated with each purification to break down the connective tissue in which the mast cells reside. The enzymes DNase, collagenase, and hyaluronidase used to digest the tissue are extremely expensive. In addition, the process is labor intensive given that the connective tissue must be cut methodically with scissors; a process that can last up to eight hours.

Mast cells from mast cell lines are also been problematic. Selective and consistent development of human mast cells first became possible when either cord blood or fetal liver mononuclear cells co-cultured with murine 3T3 fibroblasts gave rise to mast cells [4], because human Kit binds murine as well as human SCF. Later, human SCF alone proved to be a potent growth and differentiation factor for mast cells derived from fetal liver and cord blood progenitors [5]. The mast cell leukemia cell line, HMC-1 [6] and two mast cell leukemia/sarcoma cell lines, LAD 1 and 2, offer additional opportunities to study human mast cells [7]. However, these cell lines differ phenotypically from tissue-derived non-transformed mast cells. For example, HMC-1 cells, though they double every 1-2 days, lack surface FcϵRI receptors, are SCF independent and express levels of histamine and tryptase that are <1% those of tissue-derived mast cells. Two novel SCF-dependent human mast cell lines, designated LAD 1 and 2, were established from bone marrow aspirates from a patient with mast cell sarcoma/leukemia [7]. However, these cells divide extremely slowly and thus have not been widely used. Accordingly, obtaining large numbers of human mast cells to study their biology is still a laborious process and cell lines exhibit additional inadequacies for functional and biochemical studies.

Cord blood contains progenitor cells that can be used to obtain mast cells [8]. However, there are several variations in culturing conditions which appear to affect functional responses. For example, some investigators culture the cord blood progenitor cells in different cocktails of cytokines (IL-6, IL-10) in addition to the required growth factor-stem cell factor (SCF) while other laboratories do not. This has led to issues of reproducibility as the final mast cells cultures have completely different phenotypical and functional responses depending on the culture conditions.

Human embryonic stem (hES) cells represent a unique population of cells capable of self-renewal and differentiation. These cells have the potential to differentiate into nearly all cell types of the human body including neuronal, skin, adrenal, keratinocytes, cardiomyocytes, and many others [9]. For example, the hES cell line H1 has been shown to differentiate into myeloid dendritic cells and used to study these cells at different stages of maturation [10]. This cell line was also used to generate MHC class II+ leukocytes resembling dendritic cells and macrophages capable of antigen presentation. Human mast cells were shown to develop from embryonic stem cells following genetically programed differentiation into CD34+ hematopoietic progenitor cells, and stimulation by growth factors [11]. However, the use of hES cells while attractive, presents logistical and ethical problems stemming from their source, human embryos, and are not autologous making them useless for therapeutic cell immunotherapy.

None of the aforementioned mast cells could be used for autologous purposes. For mast cells to be used as a therapy they must come from the same patient for which treatment of some disorder is needed. Otherwise the immune system will reject the mast cells. This is another benefit of adipose derived mast cells as they can come from the same donor for which treatment is to be used with the mast cells.

Because of the difficulty in obtaining human mast cells in sufficient number and with high purity, previous studies largely relied on using rodent mast cells such as rat peritoneal mast cells or mouse bone marrow-derived cultured mast cells; however, rodent cells are not ideal because mast cells are heterogeneous, and there are many differences between species.

Mast cells can participate in certain inflammatory diseases and cancers. For example, mast cells can secrete proinflammatory cytokines involved in neuro-inflammatory processes and cancer. Mast cells can also accumulate in the stroma surrounding certain tumors such as breast cancer secreting molecules that can benefit tumors [12].

Mast cell granules contain chemical mediators such as histamine and cytokines, and the degranulation reaction in mast cell plays an important role in allergic diseases such as pollinosis, bronchial asthma and atopic dermatitis, and various inflammatory diseases including autoimmune diseases. The degranulation is caused by signal transduction through the IgE receptor.

4.C. Method of Obtaining Mast Cells from Adipose Derived Stems Cells

The presently disclosed subject matter provides an isolated human mast cell obtained from human adipose derived stem cells. The presently disclosed subject matter also provides a method of obtaining human mast cells, comprising separating stem cells from adipose tissue, culturing the stems cells for a period of time, increasing tumor killing molecules within the cells, and establishing single cell cultures comprising one or more colonies of human mast cells. The method of obtaining mast cells from adipose tissue is far less time consuming compared to other tissue sources, and requires less reagents and costs.

4.C.ii. Separating Stem Cells from Adipose Tissue

In one embodiment, the adipose stem cells are separated from adipose tissue. Various techniques may be utilized to separate or purify the stem cells from adipose tissue, including those disclosed in [13-15], all of which are incorporated herein by reference in its entirety. In some embodiments, stem cells are obtained from adipose tissue by mincing the adipose tissue and adding tissue digestion enzymes. The homogenate is washed and the stem cells separated using antibodies and magnetic cell sorting before culturing in conditioned medium obtained from human skin mast cell cultures. In another embodiment, the homogenate is washed and cultured in conditioned medium obtained from human skin mast cell cultures or other defined mediums.

Found in most tissues of the human body, adult stem cells can be isolated from patients using techniques of varying invasiveness [16]. The most abundant source of adult stem cells is adipose tissue. Adipose tissue is composed predominantly of adipocytes with the remainder of the cells making up what is termed the stromal vascular fraction (SVF). The SVF is formally defined as the cells making up the pellet formed upon centrifugation of enzymatically dissociated adipose tissue [16]. The exact cellular makeup of the SVF has not been defined, however it is known to contain endothelial cells, pericytes, immature immune cells, and adipose derived stem cells. Adipose derived stem cells are mesodermal stem cells that have been shown to be capable of myogeneic, osteogeneic and chondrogenic differentiation in vitro.

4.C.iii. Culturing Stem Cells and Establishing Single Cell Cultures Comprising One or More Colonies of Human Mast Cells

In one embodiment, the adipose stems cells are brought up from cryopreservation and cultured in stem cell media, and then cultured in conditioned mast cell media. In one embodiment, the adipose stems cells are cultured in the presence of conditioned mast cell media [17]. In one embodiment the conditioned mast cell media is culture medium taken from human mast cell cultures. In one embodiment, the human mast cell cultures are human skin mast cell cultures. In one embodiment, the human skin mast cell cultures comprise human skin mast cells cultured in serum-free cell medium and recombinant human stem cell factor. In one embodiment, the adipose stems cells are cultured in the presence of stem cell factor, human immunoglobin E, and conditioned mast cell media. In another embodiment, the adipose stem cells are cultured in the presence of apolipoprotein A, plasma protease C1 inhibitor, Haptoglobin OS, and Serotransferrin.

In one embodiment, the stem cells are cultured for a period of time. Culturing cells in a medium may be performed for various periods of time. The stems cells may be cultured for periods of about 5 days to 9 weeks, about 5 days to 10 weeks or more, about 5 days to 4 weeks or more, about 5 days to about 3 weeks, about 1 week to about 3 weeks, about 1-2 weeks, or about 7, 8, 9, 10, 11, 12, 13, 14 days, about 1, 2, 3, 4, 5, 6, 7, 8, 9, 10 weeks or more, or any range derivable therein.

In one embodiment, the stem cells are cultured for 4 to 5 weeks in flasks >75 cm2 and in conditioned media. One skilled in the art can select optimal containers for culturing stems cells. In one embodiment, the mast cells are separated from stem cells by pipetting such that the stem cells remain adherent and the mast cells are removed. In one embodiment the mast cells are separated from stem cells with a 5 to 10 ml pipet. The number of pipet events (going up and down) ranges from 5-10 and the force of the pipetting is dependent on the pipettor used. In another embodiment the mast cells are separated from the stem cells using cell sorting based on the different sizes of the stem cells and mast cells. In another embodiment the mast cells are separated from the stem cells using magnetic cells sorting of the stem cells using stem cell-specific cell surface markers, thus avoiding potential activation of the mast cells with mast cell-specific markers. Mast cells may be separated from stem cells using any other commonly used technique for sorting cells within a mixed population.

Various media, including both defined and undefined media (i.e., including one or more animal product such as serum), may be used with the present invention to differentiate adipose-derived stem cells into mast cells. In some embodiments, serum may be excluded from the media (i.e., “serum free” media) and protocols of the present invention. The exclusion of serum may result in an increased consistency in the results due to the elimination of variation in the contents between batches or lots of serum. Additionally, the exclusion of serum may result in the production of mast cells which, morphologically, appear to more consistently resemble mast cells in vivo. In various embodiments, serum may be replaced with X-Vivo media, which is a serum-free formulation optimized to grow and maintain undifferentiated stem cells in culture and are available from Lonza.

Various growth factors are known in the art and may be used with the present invention. In certain embodiments the adipose stem cells differentiate into mast cells in the presence of medium that has been obtained from skin derived mast cell cultures that have been in culture for greater than 1 week. In certain embodiments, a differentiation medium such as a mast cell differentiation medium of the present invention may contain one or a combination of the growth factors: nitric oxide, nutraceuticals, COX-2 inhibitors, inhibitors of intracellular cAMP. Such growth factors may increase the amount of tumor necrosis factor alpha in mast cell granules. In certain embodiments, a differentiation medium such as a mast cell differentiation medium of the present invention may contain one or a combination of the growth factors: FLT-3 ligand, stem cell factor (SCF), thrombopoietin (TPO), interleukin-3 (IL-3), Immunoglobin E, or interleukin-6 (IL-6). In certain embodiments, the growth factors are recombinant growth factors that are exogenously added to a differentiation media. The growth factor(s) included in a differentiation media may be recombinant human growth factors. Alternately, the growth factor(s) may be non-human growth factors (e.g., mammalian, etc.) or a combination of human and non-human growth factors. In certain embodiments, non-human growth factors may be advantageously used, e.g., in instances where there is a cost-savings associated with the use of the non-human growth factor as compared to the analogous human growth factor.

Stem cell factor (SCF) is a cytokine which binds CD117 (c-Kit). SCF is also known as “KIT ligand,” “c-kit ligand,” or “steel factor.” SCF exists in two forms: cell surface bound SCF and soluble (or free) SCF. Soluble SCF is typically produced in vivo by the cleavage of surface bound SCF by metalloproteases. SCF can be important for the survival, proliferation, and differentiation of progenitors and other progenitor cells. In certain embodiments, SCF is included in a culture medium of the present invention at a concentration of from about 5 to about 500 ng/ml, 25 to about 500 ng/ml, from about 25 to about 200 ng/ml, from about 50 to about 150 ng/ml, from about 25 to about 200 ng/ml, from about 75 to about 300 ng/ml, or any range derivable therein. In certain embodiments, SCF is included in the defined culture media at a concentration of about 25, 30, 35, 40, 45, 50, 55, 60, 65, 70, 75, 80, 85, 90, 95 or about 100 ng/ml.

Growth factor(s) may be included in a culture medium of the present invention at a concentration of from about 5 to about 500 ng/ml, 25 to about 500 ng/ml, from about 25 to about 200 ng/ml, from about 50 to about 150 ng/ml, from about 25 to about 200 ng/ml, from about 75 to about 300 ng/ml, or any range derivable therein.

A differentiation culture medium of the present invention may also contain additional components such as nutrients, amino acids, antibiotics, buffering agents, and the like. In various embodiments, a differentiation culture medium may contain one or more vitamin, mineral, salt, lipid, amino acid, or other component. In certain embodiments a culture medium of the present invention may contain non-essential amino acids, L-glutamine, Pen-strep, and monothioglycerol.

4.D Modification of Mast Cells

In one embodiment the mast cells can be genetically modified with a nucleic acid sequence that is native (endogenous) or foreign (heterologous) to the cells. For examples, the cells may be genetically modified to produce proteins, peptides, hormones, growth factors, and other biologically active biomolecules. The mast cells of the invention can be genetically modified to modulate (e.g., inhibit expression, inactivate, or overexpress) endogenous genes, such as the high affinity IgE receptor (FcϵR1) or CD117 (C-Kit). The mast cells of the invention can be genetically modified to modulate (e.g., inhibit expression, inactivate, or overexpress) endogenous genes, such as histamine or tryptase (downregulate) or TNF or GM-CSF (upregulate). The term “genetic modification” as used herein refers to the stable or transient alteration of the genotype of a cell of the subject invention by intentional introduction of exogenous nucleic acids by any means known in the art (including for example, direct transmission of a polynucleotide sequence from a cell or virus particle, transmission of infective virus particles, and transmission by any known polynucleotide-bearing substance) resulting in a permanent or temporary alteration of genotype. The nucleic acids may be synthetic, or naturally derived, and may contain genes, portions of genes, or other useful polynucleotides.

Exogenous nucleic acids can be introduced into a mast cell of the invention by viral vectors (retrovirus, modified herpes virus, herpes virus, adenovirus, adeno-associated virus, and the like), non-viral vectors (e.g., lipid-based or liposomal delivery), or direct DNA transfection (calcium phosphate transfection, DEAE-dextran, electroporation, and the like), for example.

In some embodiments the adipose-derived mast cells have been genetically modified with a nucleic acid sequence encoding sTRAIL, hTERT, SV-40 Large T antigen, TLX1.

In some embodiments the adipose-derived mast cells have been genetically modified using CRISPR to downregulate potentially harmful mediators such as histamine, tryptase, reactive oxygen species, VEGF, IL-8, IL-6, IL-13, TGF-beta, PAF and upregulate potentially therapeutic beneficial mediators such as TNF-alpha, heparin, IL-9, and GM-CSF.

In some embodiments, the human mast cells have been genetically modified by transduction, transfection, or electroporation. Transduction procedures include: (a) the production of lentiviral particles in HEK293 cells, and (b) the introduction of viral particles to human adipose derived stem cells.

Transfection procedures include Transfection procedures include (a) incubating DNA with transfection reagent, (b) incubation of DNA/transfection reagent complex, and (c) selection of transfected cells using G418.

Electroporation procedures include (a) adding DNA to serum-free cell culture media, (b) introducing cells in DNA/serum-free media into electroporation cuvette, and (c) applying electric field to cells.

In some embodiments the ex vivo mast cells are incubated with various agents that increase levels of tumor necrosis factor-alpha, that may be tumoricidal, such as Transfer Factor Plus, IMUPlus (non denatured milk whey protein), ascorbic acid; Agaricus Blazeii Murill tea extract, nitrogenated soy extract, or Andrographis Paniculata [125], Rofecoxib [126], nitric oxide [127], GMCSF, and IL-3 [127].

In some embodiments the ex vivo mast cells are incubated with agent(s) that increase levels of reactive oxygen species that may be tumoricidal. Reactive oxygen species that may be tumoricidal include peroxides, superoxide, hydroxyl radical, and singlet oxygen. Agents that increase levels of such reactive oxygen species levels include barley grass extract (Biomed Rep. 2017 June;6(6):681-685. doi: 10.3892/br.2017.897. Epub 2017 May 3.).

In some embodiments the ex vivo mast cells are incubated with agent(s) that increase levels of granule mediators (preformed and newly synthesized) that may be tumoricidal. Such granule mediators that may be tumoricial include IL-9, heparin, histamine, TNF, and ROS.

In some embodiments the ex vivo mast cells are incubated agent(s) that decrease levels of or inhibit granule mediators (preformed and newly synthesized) that promote tumor growth. Such granule mediators that promote tumor growth include VEGF, MMP, CxCL8, IL-6, TGF-b, adenosine, and PAF, IL-13.

4.E Compositions and Methods of Transplanting

The presently disclosed subject matter also provides a composition comprising human mast cells obtained from human adipose derived stem cells. The presently disclosed subject matter also provides a method of transplanting human mast cells comprising administering human mast cells to a mammal, wherein the mast cells have been obtained from human adipose derived stem cells. The cells can be administered locally at a desired anatomical site or systemically. In some embodiments, the cells are administered intravenously, intraperitoneally, or within a solid tumor. The cells can be administered to the mammal to destroy a tumor through the release of toxic mediators released from the mast cells granules. The cells can be administered to the mammal to treat a mast cell disorder or a disorder characterized by loss, damage, or dysfunction of mast cells, e.g., as mast cell replacement therapy. The cells can also be administered to a non-human mammal, such as an animal model of disease, for research purposes. Preferably, the mast cells administered to the mammal are at least partially purified.

The mast cells of the subject invention can be administered to a human or non-human mammal in isolation or within a pharmaceutical composition comprising the mast cells and a pharmaceutically acceptable carrier. As used herein, a pharmaceutically acceptable carrier includes solvents, dispersion media, coatings, antibacterial and antifungal agents, isotonic agents, and the like. Pharmaceutical compositions can be formulated according to known methods for preparing pharmaceutically useful compositions. Formulations are described in a number of sources that are well known and readily available to those of ordinary skill in the art. For example, Remington's Pharmaceutical Science (Martin E. W., Easton Pa., Mack Publishing Company, 19th ed.) describes formulations that can be used in connection with the subject invention. Formulations suitable for parenteral administration, for example, include aqueous sterile injection solutions, which may contain antioxidants, buffers, bacteriostats, and solutes that render the formulation isotonic with the blood of the intended recipient; and aqueous and nonaqueous sterile suspensions that may include suspending agents and thickening agents. It should be understood that in addition to the ingredients particularly mentioned above, the formulations of the subject invention can include other agents conventional in the art having regard to the type of formulation and route of administration in question.

The mast cells of the subject invention (genetically modified or not genetically modified) can be administered on or within a variety of carriers that can be formulated as a solid, liquid, semi-solid, etc. For example, genetically modified cells or non-genetically modified cells can be suspended within an injectable hydrogel composition (U.S. Pat. No. 6,129,761) or encapsulated within microparticles (e.g., microcapsules) that are administered to the mammal and, optionally, released at the target anatomical site (Read T. A. et al., Nature Biotechnology, 2001, 19:29-34, 2001; Joki T. et al., Nature Biotechnology, 2001, 19:35-38; Bergers G. and Hanahan D., Nature Biotechnology, 2001, 19:20-21; Dove A. Nature Biotechnology, 2002, 20:339-343; Sarkis R. Cell Transplantation, 2001, 10:601-607).

The mast cells can be administered to a mammal by any method of delivery, such as intratumorally, intravascularly, intracranially, intracerebrally, intramuscularly, intradermally, intravenously, intraocularly, orally, nasally, topically, or by open surgical procedure, depending upon the anatomical site or sites to which the mast cells are to be delivered. The mast cells can be administered in an open manner, as in the heart during open heart surgery. The mast cells can be infused intravenously, for example. The mast cells can be co-administered with other cell types or biologically active agents such as drugs (e.g., immunosuppressive agents).

The mast cells of the invention can be administered as autografts, syngeneic grafts, allografts, and xenografts, for example. As used herein, the term “graft” refers to one or more cells intended for implantation within a human or other mammal Hence, the graft can be a cellular or tissue graft, for example. In some embodiments, the cells are autologous (the recipient's own cells), i.e., administered as an autograft. The mast cells of the invention can be obtained from banked (e.g., frozen and stored) or unbanked adipose tissue.

4.E Cell Line Comprising Human Mast Cells Obtained from Human Adipose Derived Stem Cells

The presently disclosed subject matter also provides a cell line comprising human mast cells obtained from human adipose derived stem cells. Currently, there are limited human mast cell lines that are phenotypically and functionally similar to tissue-derived. A cell line comprising human mast cells obtained from human adipose derived stem cells may be obtained by isolating the adipose mast cells and immortalizing them through transfection or electroporation that manipulates the genetic material in such a way that they become growth factor independent (cell line) and can be cultured in perpetuity.

The practice of the present invention can employ, unless otherwise indicated, conventional techniques of molecular biology, microbiology, recombinant DNA technology, electrophysiology, and pharmacology are within the skill of the art. Such techniques are explained fully in the literature (see, e.g., Sambrook, Fritsch & Maniatis, Molecular Cloning: A Laboratory Manual, Second Edition (1989); DNA Cloning, Vols. I and II (D. N. Glover ed. 1985); Perbal, B., A Practical Guide to Molecular Cloning (1984); the series, Methods In Enzymology (S. Colowick and N. Kaplan eds., Academic Press, Inc.); Transcription and Translation (Hames et al. eds. 1984); Gene Transfer Vectors For Mammalian Cells (J. H. Miller et al. eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. eds. (1991) IRL Press)).

4.F Mast Cells Obtained from Adipose Derived Stem Cells and Sensitized with Immunoglobulin-E Targeted to a Cancer Antigen and Methods of Preparing Thereof

4.G Compositions and Methods of Treating a Tumor

The presently disclosed subject matter also provides a method of treating a tumor in a subject, comprising administering to a subject a therapeutically effective amount of mast cells obtained from adipose derived stem cells, wherein the mast cells are autologous to the subject and sensitized with immunoglobulin-E targeted to a cancer antigen. Mast cells are tissue-based immune cells that bind IgE with extremely high affinity through FcϵRI. The mast cells are targeted to cancer cells through IgE where they are hypothesized to degranulate and release TNF stores (and other mediators) to kill the tumor cells, inhibit tumor angiogenesis, tumor invasion and contribute to the composition of the immunosuppressive tumor microenvironment. Previous studies have demonstrated that high mast cell densities in breast cancer and colorectal cancer is associated with favorable prognosis suggesting mast cells have tumoricidal activity, possibly through their ability to store and release (upon FcϵRI stimulation) tumor necrosis factor alpha (TNF-α); uniquely prestored in human mast cell granules. [91, 92] To this end, it appears that even the different location of mast cells within the tumor microenvironment is prognostic, with a high intra-tumoral density being associated with a favorable prognosis and a high peri-tumoral density associated with a poor prognosis [94, 95].

Tumor necrosis factor is a multifunctional cytokine that plays important roles in cellular function. It was identified in the late 1970s as a cytokine produced by immune cells having a capacity to suppress tumor cell proliferation and induce tumor regression [72, 73]. As its name implies TNF has tumor necrotizing capabilities and has been studied as a potential therapeutic intervention for breast cancer. For example, blocking NF-κB acts with a wide variety of compounds acts synergistically in inducing TNF-cytotoxicity [74]. Similarly, concurrently suppressing NF-κB and Akt synergistically causes TNF-induced cytotoxicity in lung cancer cells [75]. Other compounds can enhance TNF-induced cell death in an NF-κB-independent manner [74]. In addition, TNF can be used as an adjuvant that enhances the anti-cancer effect of chemotherapy agents such as doxorubicin, anti-EGFR therapy, and resistance to EGFR tyrosine kinase inhibitor in lung cancer [74]. The combination of TNF and chemotherapeutic agents has been shown to be an effective therapeutic strategy for many tumors by increasing tumor sensitivity to treatment.

The biggest impediment to using TNF as an anti-cancer agent is its systemic toxicity. Thus, strategies that limit its systemic distribution through local drug-delivery systems in patients with certain types of organ-confined solid tumors have been investigated. The local administration of TNF through isolated limb perfusion in advanced melanomas and soft tissue sarcomas of the limbs was demonstrated to be safe with negligible systemic toxicity [76, 77]. This procedure has also been demonstrated to prevent loss of in patients with metastasized, bulky, limb-threatening tumors [77, 78]. Isolated limb perfusion with TNF in combination with hyperthermia or doxorubicin have also shown promising results [79, 80]. TNF-based isolated hepatic perfusion for hepatic metastases and intratumoral administration of TNF into the post-operative tumor cavity for patients with malignant glioma are also show to be safe and effective therapies [81, 82] although follow up studies did not show improvement [83]. In short tumor-targeted delivery of TNF has been used to effectively inhibit/destoy certain tumors. The major drawback is its systemic toxicity so finding ways to maximize the local concentration targeting tumor cells and subsequently minimizing the dose may hold promise in anti-tumor therapies.

Immunoglobulin-E or IgE is an antibody comprising Fc epsilon (c) constant regions and a variable region comprising at least one antigen binding region specific for an antigen (e.g. a cancer antigen). The term “antigen binding region” refers to that portion of an antibody of the invention which contains the amino acid residues that interact with an antigen and confer on the antibody its specificity and affinity for the antigen. The antibody region includes the “framework” amino acid residues necessary to maintain the proper confirmation of the antigen binding residues. In some embodiments the IgE is a monoclonal antibody, or a chimeric antibody. In one preferred embodiment, the therapeutic IgE antibody is a chimeric monoclonal antibody. The term “chimeric monoclonal antibody” refers to monoclonal antibodies displaying a single binding specificity which have one or more regions derived from one antibody and one or more regions derived from another antibody. In a preferred embodiment of the invention, the constant regions are derived from human Fc epsilon (c) (heavy chain) and human kappa or lambda (light chain) constant regions. The variable regions of a chimeric antibody may be of human or non-human origin but are typically of non-human origin. In one embodiment, the variable region is of non-human origin such as from rodents, for example, mouse (murine), rabbit, rat or hamster. In one embodiment, the variable region is of murine origin. Previously published methodology used to generate mouse/human chimeric or humanized antibodies that has yielded the successful production of various human chimeric antibodies or antibody fusion proteins (Helguera G, Penichet M L., Methods Mol. Med. 109:347-74 (2005)). Other methods for producing chimeric antibodies are well known in the art.

The use of IgE has been hypothesized to have potential in cancer therapeutics given the several potential advantages over IgG isotypes. The IgE-FcϵRI affinity is extremely high (Kα=10¹⁰ M⁻¹) which allows IgE to remain bound to immune effector cells even in the absence of antigen and has implications for immune cell stimulation [88, 92, 98]. The amount of IgE in serum compared to IgG is also much lower (<1% vs 85%) thus the competition for FcR occupancy is much lower for IgE [90, 92, 98]. Another potential advantage is that there is no known inhibitory FcϵR as there is for FcγR suggesting that IgE may escape the suppressive effects of the tumor microenvironment. Inhibitory signaling through co-aggregation of FcϵRI and FcϵRII receptors on human mast cells has been demonstrated [88]. Another advantage of IgE in the treatment of solid tumors is the IgE prolonged half-life in tissues (2 weeks compared to 2-3 days for IgG). This results in local retention of IgE by FcϵRI-expressing resident effector cells and longer immune surveillance. Lastly, the binding of IgE to FcϵRI and CD23 promotes several cell killing modes including pro-inflammatory mediators and cytokines released by mast cells and basophils that recruit professional killer cells (such as neutrophils and eosinophils) on site. In addition, IgE binding induces antibody dependent cellular cytotoxicity (ADCC) Lastly, IgE binding induces antibody dependent cellular phagocytosis (ADCP) is mediated by macrophages and monocytes resident in the tumor microenvironment [71, 89]. There are now a variety of IgE's that have been developed and studied in in vivo cancer models as shown in the following table [70].

| | IgE | | Mouse |
|---|---|---|---|
| IgE species | specificity | Targeted cancer cells | model |
| Murine | gp36 of | H2712 murine mammary | C3H/HeJ |
| | MMTV | carcinoma (s.c. and i.p.) | |
| Rat/human | Murine | E3 murine thymoma | C57BL/6 |
| chimeric | Ly-2 | (s.c.) | |
| Murine | DNP | MC38 murine colon | C57BL/6 |
| | | carcinoma cells | |
| | | expressing human | |
| | | CEA (s.c.)^{a} | |
| Murine | DNP | TS/A-LACK murine | BALB/c |
| | | mammary carcinoma | |
| | | cells coated with | |
| | | DNP (s.c.) | |
| Murine and | Colorectal | Human COLO 205 | SCID |
| murine/human | cancer | (s.c.) | |
| chimeric | antigen | | |
| Rat/human | Murine | E3 murine thymoma | NOD-SCID |
| chimeric | Ly-2 | (i.p.) | |
| Mouse/human | FBP | IGROV-1 human ovarian | C.B-17 |
| chimeric | | carcinoma cells (s.c.) | scid/scid |
| | | HUA patient-derived | nu/nu |
| | | ovarian carcinoma (i.p.) | |
| Mouse/human | NIP | TS/A-LACK murine | Human |
| chimeric | | mammary carcinoma | FcεRIα |
| | | cells coated with | transgenic |
| | | NIP (s.c.) | BALB/c |
| Human | HER2/neu | D2F2/E2 murine | Human |
| | | mammary carcinoma | FcεRIα |
| | | cells expressing | transgenic |
| | | human HER2/neu (i.p.) | BALB/c |

The first clinical trial is currently underway in patients with advanced solid tumors to examine the safety of a mouse/human chimeric IgE antibody (MOv18 IgE), specific for the tumor-associated antigen folate receptor alpha, which has exhibited superior anti-tumor efficacy for IgE compared with IgG in animal models (www.clinicaltrials.gov; clinical trial number NCT02546921, [69]).

The presently disclosed methods of treating a tumor in a subject, comprising administering to a subject a therapeutically effective amount of mast cells obtained from adipose derived stem cells, wherein the mast cells are autologous to the subject and sensitized with immunoglobulin-E targeted to a cancer antigen, provide the advantage of precise tumor targeting and controlled TNF-α release in the tumor milieu upon FcϵRI-IgE crosslinking.

The mast cells obtained from adipose derived stem cells may be sensitized with immunoglobulin-E targeted to a cancer antigen. The term “cancer antigen” as used herein can be any type of cancer antigen known in the art. The cancer antigen may be an epithelial cancer antigen, (e.g., breast, gastrointestinal, lung), a prostate specific cancer antigen (PSA) or prostate specific membrane antigen (PSMA), a bladder cancer antigen, a lung (e.g., small cell lung) cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, or a colorectal cancer antigen. Other cancer antigens include but are not limited to mucin-1 protein or peptide (MUC-1) that is found on all human adenocarcinomas: pancreas, colon, breast, ovarian, lung, prostate, head and neck, including multiple myelomas and some B cell lymphomas; mutated B-Raf antigen, which is associated with melanoma and colon cancer; human epidermal growth factor receptor-2 (HER-2/neu) antigen; epidermal growth factor receptor (EGFR) antigen associated lung cancer, head and neck cancer, colon cancer, colorectal cancer, breast cancer, prostate cancer, gastric cancer, ovarian cancer, brain cancer and bladder cancer; prostate-specific antigen (PSA) and/or prostate-specific membrane antigen (PSMA) that are prevalently expressed in androgen-independent prostate cancers; is Gp-100 Glycoprotein 100 (gp 100) associated with melanoma carcinoembryonic (CEA) antigen; carbohydrate antigen 10.9 (CA 19.9) related to the Lewis A blood group substance and is associated with colorectal cancers; and a melanoma cancer antigen such as MART-1.

An “antigen” is a molecule or portion of a molecule capable of being bound by an antibody which is additionally capable of inducing a subject to produce antibody capable of binding to an epitope of that antigen. In one embodiment, the antigen is capable of being bound by an IgE antibody of the invention to form an immune complex that is capable of inducing a specific IgE-mediated immune response to the antigen in a patient capable of mounting such immune response. As used herein, a “patient capable of mounting (the referenced) immune response” is a subject such as a human patient or other animal subject with functional T-cells, mast cells, basophils, eosinophils, monocytes, macrophages and dendritic cells with receptor affinity for the administered IgE antibody of the invention as distinguished from non-human animal models, for example, whose immune systems do not contain Fc epsilon receptors capable of binding human IgE permitting generation of functional T-cells, mast cells, eosinophils and dendritic cells in response to the administered antibody.

In some embodiments, the mast cells comprise increased levels of tumor necrosis factor-alpha. The amount of TNF-alpha in the mast cells may be increased during ex vivo culture using various cytokines, pharmaceutical agents, siRNA, nutriceuticals, or other compounds known in the art.

In some embodiments, the mast cells are genetically modified mast cells. Mast cells may be genetically modified by transduction, transfection, or electroporation, and as otherwise described herein or known in the art. In some embodiments, the genetically modified mast cells are modified to benefit tumor destruction. In some embodiments, the genetically modified mast cells comprise increased levels of tumor necrosis factor-alpha or tumor necrosis factor-alpha mRNA, or decreased levels of histamine or histamine mRNA.

### 9. EXAMPLES

The following Examples further illustrate the disclosure and are not intended to limit the scope. In particular, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims. Various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The examples set forth herein relates generally to therapeutic mast cells and compositions thereof, and methods of treating a tumor comprising administering autologous mast cells from adipose tissue. In particular, the ADMC, sensitized with a humanized anti-HER2/neu IgE, bound to and were activated by HER2/neu-positive human breast cancer cells (SK-BR-3). ADMC sensitized in this way were observed to induce apoptosis in breast cancer cells when co-cultured. Breast cancer cell apoptosis was also observed after addition of media containing mediators released from activated ADMC.

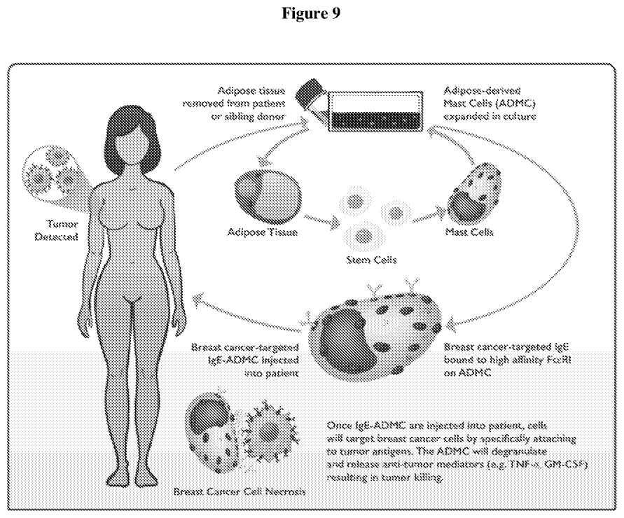

Use of autologous (or allogeneic) MC in cancer immunotherapy as illustrated in FIG. 9 has several advantages. First, mature, functional, autologous or allogeneic MC can be obtained in quantities necessary for patient infusion (approximately 10⁸-10⁹ ADMC from 50 grams of adipose tissue). Second, the availability of IgE antibodies with human constant regions (chimeric, humanized, and fully human) targeting tumor antigens has grown substantially [90, 98]. Third, the high affinity binding between IgE and FcϵRI is very stable with a long half-life resulting in an effective arming of MC, which would be able to target the tumor and so doing induce tumor cell death. The presence of dead tumor cells would facilitate their uptake and presentation of tumor antigens by antigen presenting cells (APC), eliciting an adaptive broad-spectrum anti-tumor immunity. This would increase due to MC local release of GM-CSF [22, 117] and potentially the release of suppressors of regulatory T-cells (Tregs) function as reported for IgE degranulation in murine MC [68]. Lastly, unlike other immune cells currently being used for cancer immunotherapy [64], MC loaded with IgE are equipped to kill tumor cells without prior sensitization or genetic reprogramming, which is time consuming and expensive.

I. Materials and Methods: Adipose-Derived Mast Cells

I.A. Media

The isolation media comprises: lx Hanks Balanced Salt Solution (ThermoFisher, 14025092), 1% Fetal Bovine Serum (Worldwide sci, 61211144), Sodium Bicarbonate (Sigma, S5761), HEPES Buffer (Sigma, H0887), Ampho B (Sigma, A2948), Penicillin-Streptomycin (Sigma, P4333), and water.

The digestion buffer comprises: isolation media, and 0.1% Collagenase Type IA from Clostridium histolyticum (Sigma-Aldrich, 234153).

The adipose-derived stem cell media comprises: DMEM w/ 4.5 g/L Glucose (Worldwide sci, 61211056), 10% Fetal Bovine Serum, 1% L-Glutamine (Sigma, G7513), 1% HEPES Buffer, and 1% Penicillin/Strepomycin.

The complete mast cell media comprises: conditioned X-vivo 15 (Lonza, 04-418Q) with Stem Cell Factor (SCF), 100 ng/mL SCF (Sigma, S7901), 10 ng/mL Human IgE (Sigma, AG30P), and wherein all components are filtered through 0.22 μm pore filters (Worldwide sci, 51101372) to remove cells and contaminants.

Alternatively, the complete mast cell media comprises: conditioned X-vivo 15 (Lonza, 04-418Q) with 20-100 ng/ml Stem Cell Factor (SCF). In other embodiments the media used for differentiation is media obtained from skin mast cell cultures (CMCM; Zhao, Kepley, C. L Morel, P. A. Okumoto, L. M. Fukuoka, Y. Schwartz, L. B; 2001 J Immunology). In other embodiments the media used for differentiation contains 1-10 ng/mL Human IgE (Sigma, AG30P). In other embodiments the differentiation media contains all SCF and IgE and media from skin mast cell cultures.

I.B. Adipose Derived Stem Cell Isolation

Liposuction aspirate is obtained from the surgical center within one hour of liposuction procedure. 30 mL aliquots of adipose tissue are placed into 50 mL conical tubes (ThermoFisher, AM12502), which are then filled to 45 mL of digestion buffer. Tubes are placed into a 37° C. orbital shaker for one hour with constant agitation at 150 rpm. Upon completion of digestion, the cell slurry is centrifuged at 360×g for 20 minutes. During the centrifugation, fat laden adipocytes and free lipids float to the top of the tube and form a distinct layer. Following centrifugation, this layer is then removed with a 10 mL serological pipette (ThermoFisher, 170367). The remaining cell pellet is then washed with fresh Isolation media, passed through a 100 pm cell strainer (Greiner bio-one, 542 000), resuspended in ADSC Media, plated in T25 cell culture flasks (Worldwide sci, 50051665) and grown in a 37° C. incubator with 5% CO2.

In another exemplary method, skin and adipose tissue was obtained from patients undergoing cosmetic surgery. Adipose tissue is incubated with Hanks' balanced salt solution (HBSS), 1% fetal bovine serum, 0.04% sodium bicarbonate, 1% HEPES, 0.5% amphotericin B, 1% streptomycin/penicillin and 0.1% collagenase type 1A. Cells are placed into a 37° C. orbital shaker for one hour with constant agitation at 150 rpm. The cell slurry is centrifuged at 1,345 rpm for 15 minutes and adipocytes washed, suspended in medium (DMEM with 4.5 g/L glucose, 10% fetal bovine serum, 1% streptomycin/penicillin, 1% L-glutamine and 1% HEPES), and cultured for up to four days or a week before adding MC-differentiating media.

I.C. Mast Cell Differentiation

Conditioned mast cell media (CMCM) is produced using media from primary cultures of human skin mast cells hSMCs are cultured in X-Vivo 15 (Lonza), with 40 or 80 ng/mL Recombinant Human Stem Cell Factor. Every seven days, half of the media is removed and replaced with fresh media. The Conditioned media is collected and filtered through a 0.22 pm filter to remove any hSMCs present and 100 ng/mL SCF and 0.1 μg/mL human IgE added. The ADSC's are cultured for one week in T-25 flasks, washed with CMCM, and placed in CMCM. Media is replaced once every week, with used media (including mast cells) being re-plated in 24 well plates for further culture.

In another exemplary method, conditioned MC media was produced using media from primary cultures of human skin MC [87] starting after five weeks in culture. Approximately every seven to ten days, half of the media was collected and replaced with fresh media. Mature ADMC can be observed after four to five weeks in culture and continue to multiply up to four months.

I.D. CD45 Depletion

While not required, in one embodiment the CD45+ non stem cells are removed before placing the remaining stem cells in stem cell medium. ADSCs were separated based on their expression of the common leukocyte antigen, or CD45. Briefly, Dynabead® Protein G beads (ThermoFisher Scientific) were conjugated with mouse Anti-Human CD45 IgG1. Beads and antibody were dialyzed against 1× PBS at room temperature overnight to remove Azide buffer. An aliquot of beads were then mixed with anti-CD45 in 2 mL MACS Buffer (lx PBS pH 7.2, 0.5% BSA, 2 mM EDTA) and incubated on ice for one hour with intermittent agitation. Following incubation, beads were washed in MACS buffer and resuspended in 1 mL MACS buffer.

ADSCs were removed from flasks using Corning™ CellStripper Dissociation Reagent, pelleted and resuspended in 500 μL of MACS Buffer. Cells were incubated with MACS buffer at a 1:2 ratio (500 μL cells: 1 mL Beads) for one hour on ice. Following incubation, the cell-bead slurry was applied to an equilibrated LS MACS columns in a SuperMACS™ Separator. The column was washed 3 times with 3 mL of MACS buffer. Filtrate was collected in a 15 mL centrifuge tube and designated “Depleted”. This process was performed twice. Following depletion, 5 mL of MACS buffer was applied to the column, the column was removed from the magnetic field and the column was flushed into another 15 mL centrifuge tube using the supplied plunger, this filtrate was designated “Enriched”. Separated cells were then pelleted, resuspended in CMCM and plated in T-25 flasks.

I.E. Histochemistry

ADMCs were collected by removing media from flasks and centrifuging them to pellet the cells. Pelleted cells were resuspended in 200 μL of media, applied to CytoSpin funnels and spun for 5 mins at 600 rpm. Cytospins were used for toluidine blue staining as described [20]. For immunohistochemistry with mast cells-specific markers slides were fixed in methanol for one hour, washed twice with 1X Tris Buffered Saline with 0.1% Triton X (TTBS), and blocked with NGS. Primary mouse anti-human antibodies to tryptase (a gift from Lawrence Schwartz, VCU), chymase (Gift from Dr. L Schwartz), or non-specific mouse IgG control (MOPC) (Sigma-Aldrich) were added (1 ug/ml) overnight in dark, humid chambers at 4° C. Cells were washed twice with TTBS and incubated with Cy3-conjugated goat anti-mouse antibodies (Jackson ImmunoResearch) for two hours along with Hoechst 33342 for nuclear staining [37, 97]. Cells were then washed and mounted in mounting media and imaged using a Zeiss AXIO Observer Z1 Spinning Disc Confocal Microscope.

I.F. Beta-Hexosaminidase, GM-CSF Release, Degranulation, and Cytokine Production from ADMC

ADMC were tested to assess functional responses to IgE and non-IgE mediated activators. For IgE-mediated activation, ADMC and HSMC were incubated with 1 μg/mL of anti-FceRI antibody for 30 min (degranulation) or overnight and beta-hexosaminidase activity or GM-CSF production were measured as described [21]. To assess non-IgE-mediated functional responses ADMC were incubated with poly-L lysine, f-Met-Leu-Phe, A23187 or LPS [22].

To further assess ADMC functional responses mediated through FcϵRI, ADMC were incubated with 1 μg/ml of anti-FcϵRI antibodies or anti-NP IgE followed by NP-BSA for 30 minutes (degranulation) or overnight (cytokines) and β-hexosaminidase release and TNF-c and GM-CSF production were measured as described [62, 119, 172]. ADMC were incubated with poly-L-lysine, f-Met-Leu-Phe (FMLP), A23187 or LPS to evaluate the non-IgE-mediated functional response [22]. For comparison, skin-derived human MC were run in parallel [34]. In some experiments anti-HER2/neu IgE sensitized ADMC challenged with ECD^{HER2} [145] or heat-inactivated serum from patients with HER/neu positive breast cancer (Cureline, Brisbane, Calif.:

| | | | | | TNM | TNM |
|---|---|---|---|---|---|---|
| | | | Pathological | | Staging | Staging |
| Serum | Diagnosis | Age | Diagnosis | Grade | (T) | (N) |
| Patient 1 | Breast | 64 | Infiltrative | G3 | T2 | N1 |
| | Carcinoma | | ductal | | | |
| | | | carcinoma | | | |
| Patient 2 | Breast | 35 | Infiltrative | G1 | T1 | N0 |
| | Carcinoma | | introductal | | | |
| | | | carcinoma | | | |

| | TNM | | | |
|---|---|---|---|---|
| | Staging | | HER2/neu | |
| Serum | (M) | Stage | Status | Treatment History |
| Patient 1 | M0 | IIB | 2+ | None |
| | | | | (treatment-naïve) |
| Patient 2 | M0 | IA | 3+ | None |
| | | | | (treatment-naïve) |

All experiments were performed in duplicate from at least two separate donors and significance (p<0.05) tested using the Student t-test.

I.G. Gene Expression

RNA was extracted from ADMC using the Qiagen RNeasy Plus Mini kit. Reverse Transcriptase PCR was performed using the Qiagen OneStep RT-PCR kit using the following primers: beta actin Forward (SEQ ID NO: 1), beta actin Reverse (SEQ ID NO: 2), 18s Forward (SEQ ID NO: 3), 18s Reverse (SEQ ID NO: 4), Tryptase Forward (SEQ ID NO: 5), Tryptase Reverse (SEQ ID NO: 6), Chymase Forward (SEQ ID NO: 7), Chymase Reverse (SEQ ID NO: 8), c-KIT Forward (SEQ ID NO: 9), c-KIT Reverse (SEQ ID NO: 10), FcϵR1a Forward (SEQ ID NO: 11) and FcϵR1a Reverse (SEQ ID NO: 12). Cycling conditions were: 50° C. for 30 mins, 95° C. for 15 mins, followed by 35 cycles of 94° C. for 45 sec, 53-63° C. for 45 sec (according to primer Tm), 72° C. for 1 min and a final 10 min extension at 72° C.

II. Results: Phenotypic and Functional Characterization of ADMC as Compared to Mast Cells Derived from Skin

II.A. Phenotypic characterization of ADMC

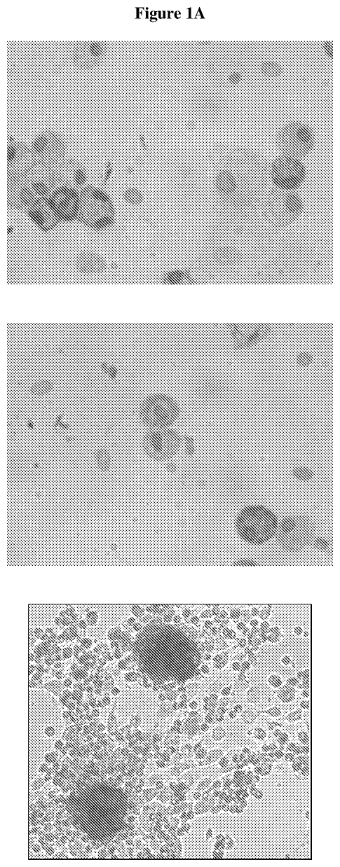

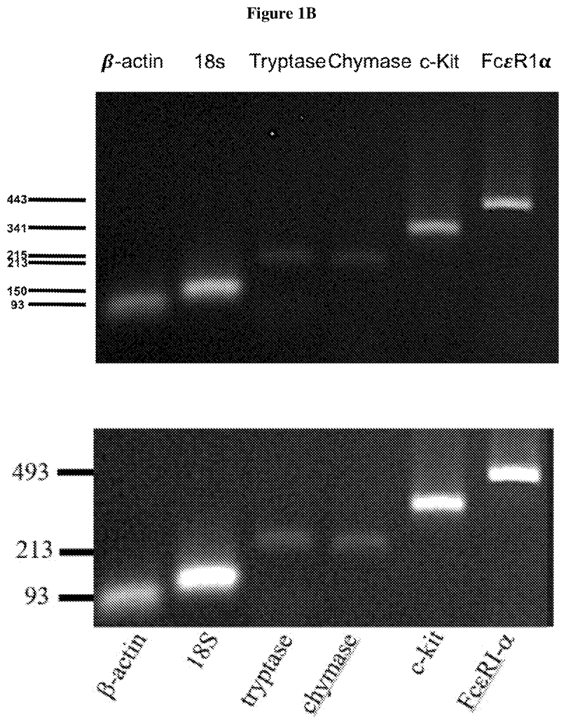

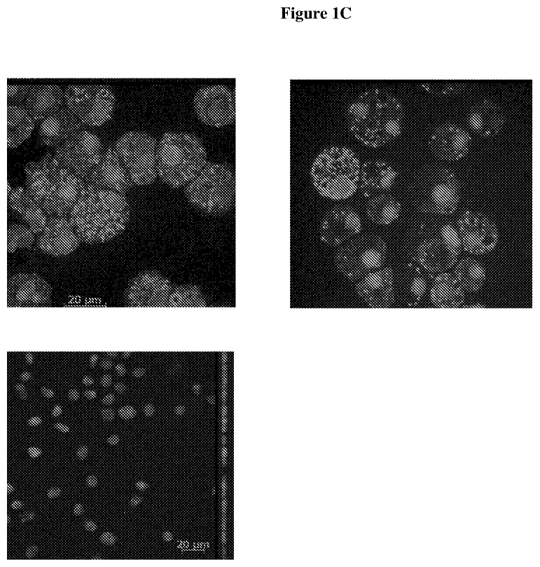

To test the hypothesis that adipose tissue contains progenitor stem cells that could be induced to differentiate into functional mast cells, adipose derived stem cells were incubated with conditioned media described above. In conditioned medium, ADMC were observed to emerge from large clumps of cells or tissue (FIG. 1A, bottom). After 4 weeks the cells differentiated into mast cells that were phenotypically similar to human skin derived human mast cells (FIG. 1A top and middle, respectively). Gene expression in ADMCs was analyzed for four important mast cell-specific genes, Tryptase, Chymase, c-kit and FcαR1α (FIG. 1B). Gene expression was measured using RT-PCR on total RNA extracted from cultured ADMCs. Beta actin and 18s ribosomal subunit primers were used as housekeeping controls. As seen in FIG. 1B (top and bottom), ADMCs showed expression of all four transcripts. Also, adipose derived mast cells are similar in size to normal human skin derived mast cells as demonstrated by the data in the Table below:

| | |
|---|---|
| Mean | 20.14407407 |
| Std Dev | 2.880001682 |
| Median | 19.67 |

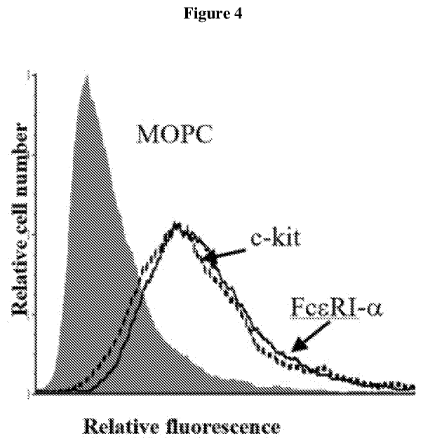

Both are close to the average size reported for human mast cells [18]. As seen in FIG. 1C, ADMC, antibodies to two major mast cell proteases, tryptase (top left) and chymase (top right) showed intense granule staining compared to isotype-matched controls (bottom). Mast cell tryptase and chymase expression was analyzed by immunohistochemistry using ADMC cytospins. Anti-tryptase (top left), anti-chymase (top right), or isotype control (bottom) antibodies were incubated overnight. The next day cytospins were washed and incubated with Cy3-labled secondary antibodies and Hoerst dye (for blue nuclei) and visualized using confocal microscopy as described in the methods herein. Thus, the ADMC are phenotypically similar to human mast cells derived from connective tissue based on these characteristics. Furthermore, the ADMC expressed surface markers for tissue MC including FcϵRI and the receptor for SCF, c-kit (FIG. 4).

II.B. Functional Characterization of ADMC

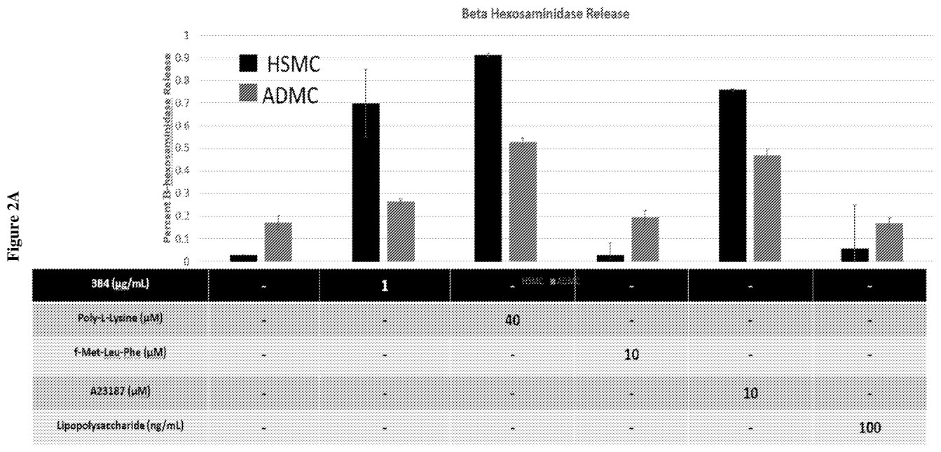

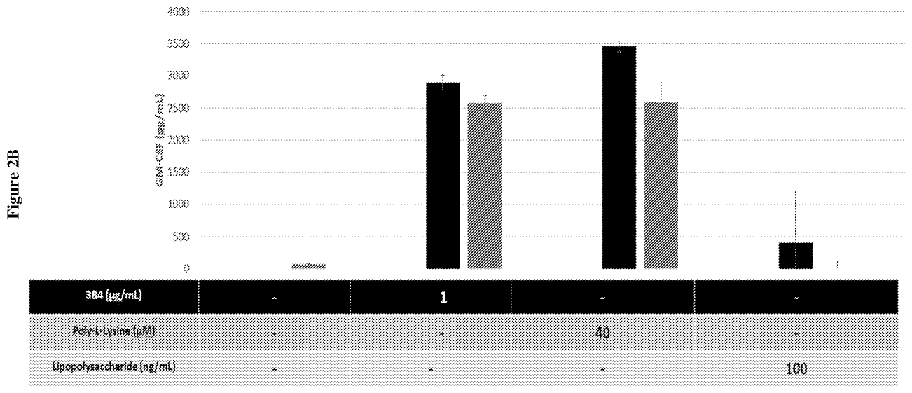

Functional response of ADMCs compared to skin derived mast cells was investigated. As seen in FIG. 2, ADMCs responded to FcϵRI-dependent degranulation (FIG. 2A) and cytokine production (FIG. 2B). ADMC demonstrated an average 43% release of beta-hexosaminidase while human skin mast cells (75%). As seen in FIG. 2B, GM-CSF and TNF-a production from ADMC and HSMC showed similar FcϵRI-mediated responses. In addition, ADMC showed non-FceRI-mediated response to poly-L-lysine, fMLP, A23187 and LPS similar as has been reported by HSMC [2].

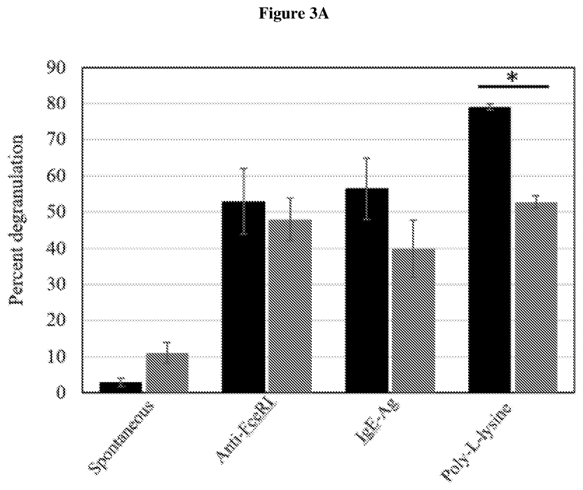

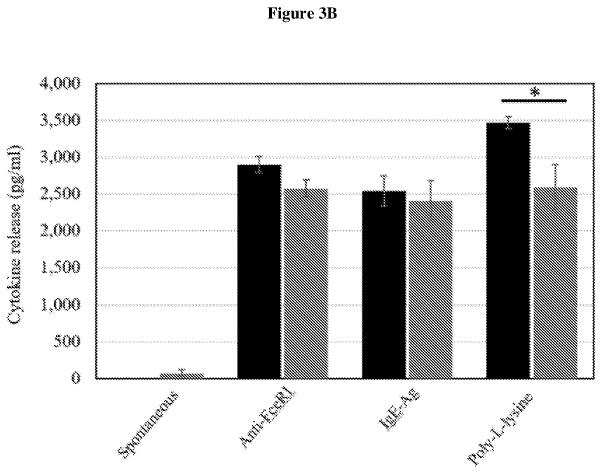

As seen in FIGS. 3A and 3B, the same data set shows ADMC responded to FcϵRI-dependent and FcϵRI-independent degranulation (FIG. 3A) and cytokine production (FIG. 3B). As previously reported [21], secretagogues that bypass FcϵRI induced cellular degranulation in both cells types. Cytokine production by ADMC and skin MC was similar in response to FcϵRI-dependent stimuli averaging 2,850 pg/ml and 2,600 pg/ml of GM-CSF in ADMC and skin MC, respectively. A similar response between skin-derived MC and ADMC was observed using non-FcϵRI-dependent stimuli poly-L-lysine (FIGS. 3A and 3B) as well as A23187, LPS, and FMLP (data not shown). Taken together, the ADMC are functionally similar to skin-derived MC in response to FcϵRI-dependent and FcϵRI-independent stimuli.

II.C. CD45 Depletion

In order to begin to elucidate the cell of origin of ADMCs, one week old cultures of ADMCs were separated using magnetically labeled antibodies based on expression of the common leukocyte antigen CD45. Following magnetic separation, CD45 positive and negative populations of ADMCs were cultured in differentiation media. Following three weeks in culture with mast cell differentiation media, mast cells were visible in the CD45 negative population, whereas the CD45 enriched population produced no observable mast cells (not shown).

Magnetic separation of SVF cells using anti-CD45 labeled beads showed that the cell of origin is CD45−, however within this subpopulation the exact identity remains unclear. Cells produced by CD45 depleted cells were shown to express tryptase and chymase, but other tests were not performed. More investigation is required in order to determine the exact cell type of origin and mechanism of differentiation, as these are currently unknown.

III. Materials and Methods: Sensitized Human Adipose-Derived Mast Cells

III.A. Sensitized Human Adipose-Derived Mast Cells with anti-HER2/neu IgE and the Kinetics of Release in Response to HER2/neu Antigen.

It was hypothesized that anti-HER2/neu IgE-sensitized adipose-derived mast cells become activated upon exposure to dimerized or tumor bound HER antigen. This is a fundamental necessity for this approach to work as the release of TNF in the granules is mediated through FcϵRI signaling pathways activated through crosslinking IgE on the surface. Here the kinetics of release induced by HER2 antigen from IgE anti-HER sensitized mast cells are assessed and mediator release measured.

Anti-HER2/neu IgE Antibodies and the Extracellular Domain of HER2/neu (ECD^{HER2})

The fully human anti-human HER2/neu IgE/kappa antibody containing the variable regions of the human scFv C6MH3-B1 has been previously described [88]. The anti-human HER2/neu IgE/kappa containing the variable regions of the humanized IgG1 antibody trastuzumab (Herceptin®) was made by subcloning the variable regions of trastuzumab [143] into the human epsilon/kappa expression vectors use to the develop the C6MH3-B1 IgE. The trastuzumab IgE and C6MH3-B1 IgE bind different epitopes of human HER2/neu. They were expressed in murine myeloma cell lines and purified as described [88].

Breast Cancer Cell-Induced Mediator Release from ADMC

ADMC were sensitized with or without 1 μg/ml of anti-HER2/neu IgE or non-specific psIgE as above and added to SK-BR-3 cells for one hour in 24 well plates. The ratio of MC to breast cancer cells varied from 1:10 to 10:1 ADMC to SK-BR-3 breast cancer cells and mediators assessed in the supernatants.

III.B. Human Adipose Derived Mast Cells, Sensitized with anti-HER2/neu IgE, Can Immuno-Detect HER2/neu-Positive Breast Cancer Cells, Degranulate, and Kill the Tumor Cells In Vitro.

Mast cells are unique in that they contain prestored, biologically active tumor necrosis factor-alpha which is immediately released upon FcϵRI/IgE co-crosslinking. Increased numbers of mast cells in breast cancer tissue correlates with a favorable prognosis. It was hypothesized that adipose-derived mast cells, sensitized with anti-HER2/neu IgE, would bind to, degranulate, and kill breast cancer cells in vitro. This killing would be mediated by the TNF-a found in the mast cell granules. Since the HER/neu antigen is on the cellular surface on breast cancer, and not in the serum, the mast cells sensitized with HER/neu IgE were hypothesized not to degranulate until it reaches the breast cancer cells. Indeed the anti-HER2/neu IgE did not induce cross-linking of the FcϵRI when complexed with shed soluble antigen ECD^{HER2} [89] consistent with the interaction of ECDHER2 with the anti-HER2/neu IgE, which is expected to be mono-epitopic in nature and the fact that ECD^{HER2} does not form homodimers in solution [103].

HER2/neu IgE-mediated binding of ADMC to breast cancer cells

To assess the ability of anti-HER2/neu IgE sensitized ADMC to bind to HER2/neu expressing SK-BR-3 breast cancer cells, confocal imaging was used on differentially labelled, live cells. The ADMC (1.5×10⁵) were sensitized with 1 μg/ml of anti-HER2/neu IgE antibodies or non-specific IgE (psIgE; provided by Dr. Andrew Saxon, UCLA) followed by the addition of MitoTracker™ Green (500 nM; ThermoFisher Scientific). The ADMC were washed once in warm media and added to the adherent, human HER2/neu-positive SK-BR-3 cells that were pre-stained with MitoTracker™ Red (500 nM; ThermoFisher Scientific) in a live cell incubator affixed to a confocal microscope and images acquired over six hours.

HER2/neu IgE-Mediated Killing of Breast Cancer Cells by ADMC and Supernatants from Activated ADMC.

To assess the ability of anti-HER2/neu IgE sensitized ADMC to induce cell death of HER2/neu expressing breast cancer cells, confocal imaging of live cells was used on differentially labelled cells. ADMC (1.5×10⁵) were sensitized with 1 μg/ml of anti-HER2/neu IgE or psIgE for two hours. Breast cancer cells (5×10⁴) on coverslips were labelled with MitoTracker™ Green (2 μM, ThermoFisher Scientific) for one hour. The washed ADMC were labelled with CellTracker™ Deep Red (which stains the cells reddish/purple under confocal; 2 μM) for one hour, washed, and added to SK-BR-3 in medium containing optimal concentration of propidium iodide (PI; which stains the cells red; 25 μg/ml) which is used to detect dead cells [146]. Images were acquired using a 561 nm laser set at 3% intensity. In parallel, cytospins of cells were made and used for immunofluorescence detection of apoptosis described above. In separate experiments, cell free supernatants from optimally activated ADMC (1.3×10⁶) through FcϵRI (1 μg/ml for 24 hours; 60%-70% release) were directly added to MitoTracker™ Green-labelled SK-BR-3 cells (5×10⁴) and cell death was monitored over time through the quantification of PI as above.

Cytochemistry and Immunofluorescence

For detection of ADMC-induced apoptosis of SK-BR-3 cells cytospins were incubated with 1 μg/ml Alexa Fluor^{TM} 488 dye (ThermoFisher Scientific) labelled mouse anti-human tryptase (1 μg/ml; for ADMC detection; green) along with Alexa Fluor™ 647 labelled mouse anti-human caspase 3 (1 μg/ml; for SK-BR-3 detection; red) or Alexa Fluor™ 647 labelled isotype control for the caspase 3 antibody.

IV. Results: Sensitized Human Adipose-Derived Mast Cells Binding, Activation, and Ability to Induce Cancer Cell Death

IV.A. Anti-HER2/neu IgE Mediates ADMC Binding to SK-BR-3 Breast Cancer Cells

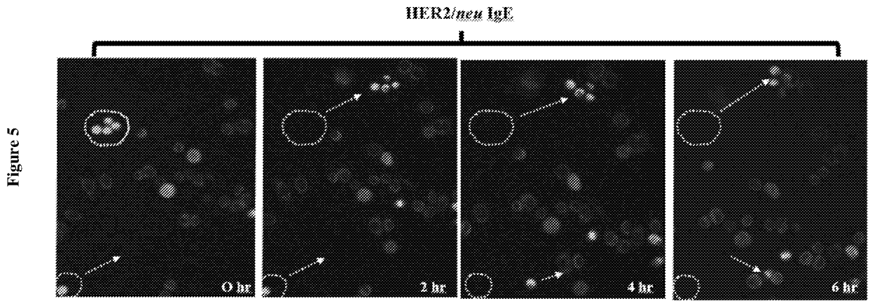

The ability of IgE HER2/neu sensitized ADMC to bind to HER2/neu-positive SK-BR-3 breast cancer cells was investigated. As seen in FIG. 5, the IgE HER2/neu-sensitized ADMC (green) bound to HER2/neu-positive SK-BR-3 breast cancer cells (red) as demonstrated in the time lapse pictures. However, ADMC sensitized with a non-specific IgE did not target the SK-BR-3 cells (data not shown). These results demonstrate that the anti-HER2/neu-IgE triggers binding of ADMC to HER2/neu-positive breast cancer cells.

IV.B. Anti-HER2/neu IgE-Sensitized ADMC Become Activated Through FcϵRI Upon SK-BR-3 Breast Cancer Cell Binding

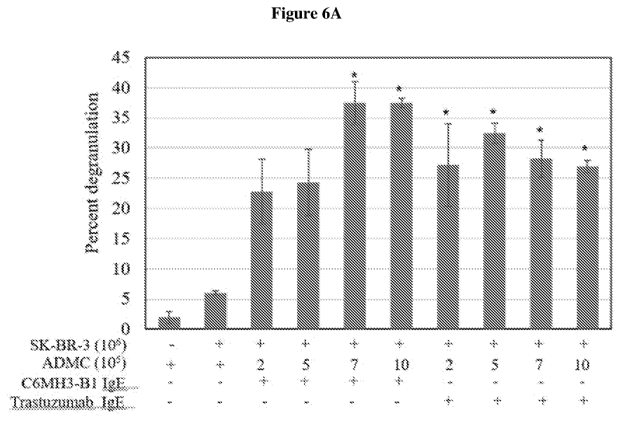

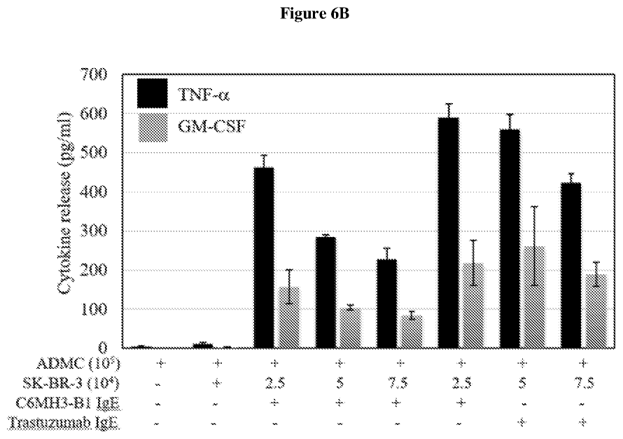

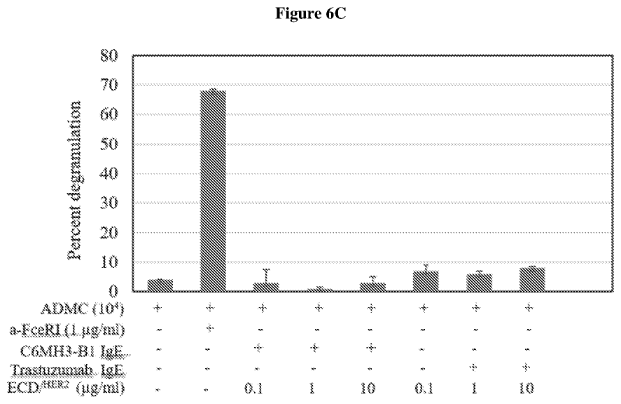

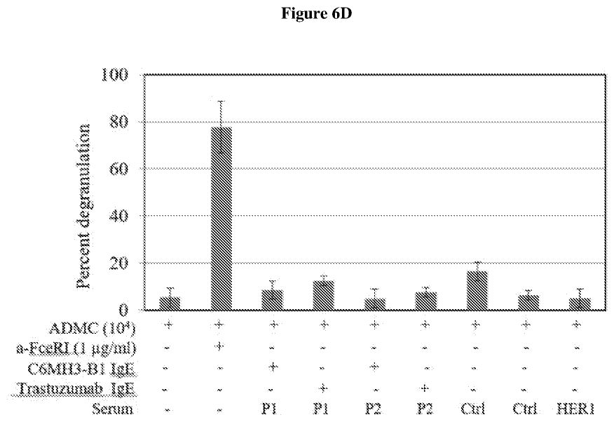

ADMC must release their mediators upon FcϵRI challenge at the site of the tumor to be effective anti-tumor agents. Thus, the ability of IgE HER2/neu sensitized ADMC to bind to HER2/neu-positive SK-BR-3 breast cancer cells and induce ADMC mediator release was investigated. ADMC were sensitized with two humanized HER2/neu IgE antibodies recognizing different epitopes (trastuzumab IgE and/or C6MH3-B1 IgE). Varying ADMC cell numbers were incubated with SK-BR-3 breast cancer cells and mediator release assessed in the medium. As seen in FIG. 6, ADMC sensitized with anti-HER2/neu IgE induced significant (p<0.05) mediator release through FcϵRI when co-incubated with the HER2/neu-positive SK-BR-3 breast cancer cells. The ADMC degranulated to release pre-formed mediators (FIG. 6A), as well as newly formed mediators TNF-α and GM-CSF (FIG. 6B).

The above results suggest the possibility of using ADMC armed with IgE antibodies as a potential cancer immunotherapy. However, a concern of the systemic administration of an ADMC sensitized with HER2/neu IgE is the possible induction of an anaphylactic reaction as patients with HER2/neu breast cancer can have elevated levels of circulating HER2 in the blood [148, 149]. The IgE antibodies are not expected to induce FcϵRI cross-linking when complexed with soluble antigen (ECD^{HER2}), given its mono-epitopic nature of this interaction and the fact that ECD^{HER2} does not form homodimers in solution [150, 151]. To address this concern, the ability ECD^{HER2} to induce FcϵRI-mediator release was examined As described previously [88], ECD^{HER2} in the presence of the anti-HER2/neu IgE antibodies did not induce degranulation, while anti-FcϵRI antibodies induced release (FIG. 6C). Furthermore, serum from two separate HER2/neu positive breast cancer patients did not induce ADMC degranulation (FIG. 6D). These results suggest that the anti-HER2/neu IgE-sensitized ADMC will not induce an anaphylactic response in vivo and only release mediators upon encountering HER2/neu on breast cancer cells.

IV.C. Co-Incubation of ADMC with SK-BR-3 Induce SK-BR-3 Cell Death Upon IgE HER2/neu-Mediated Binding

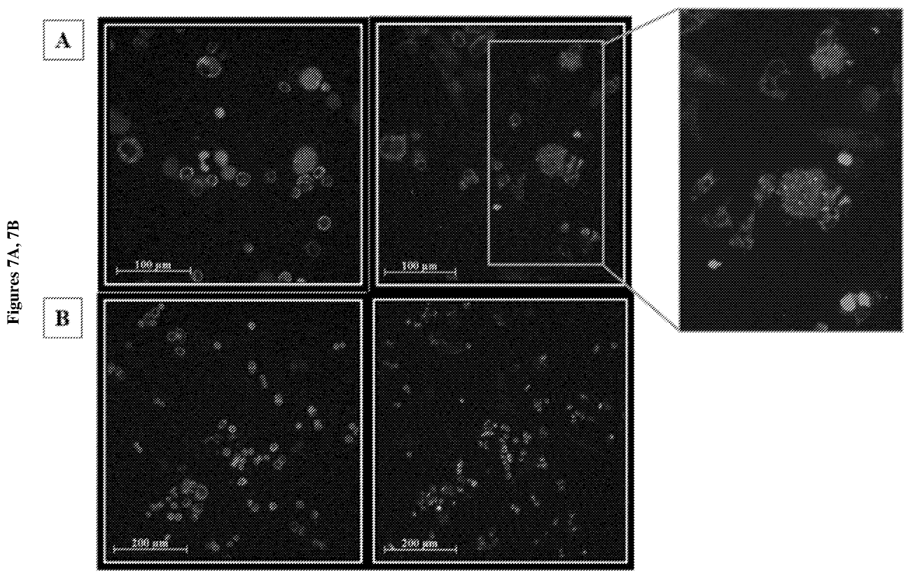

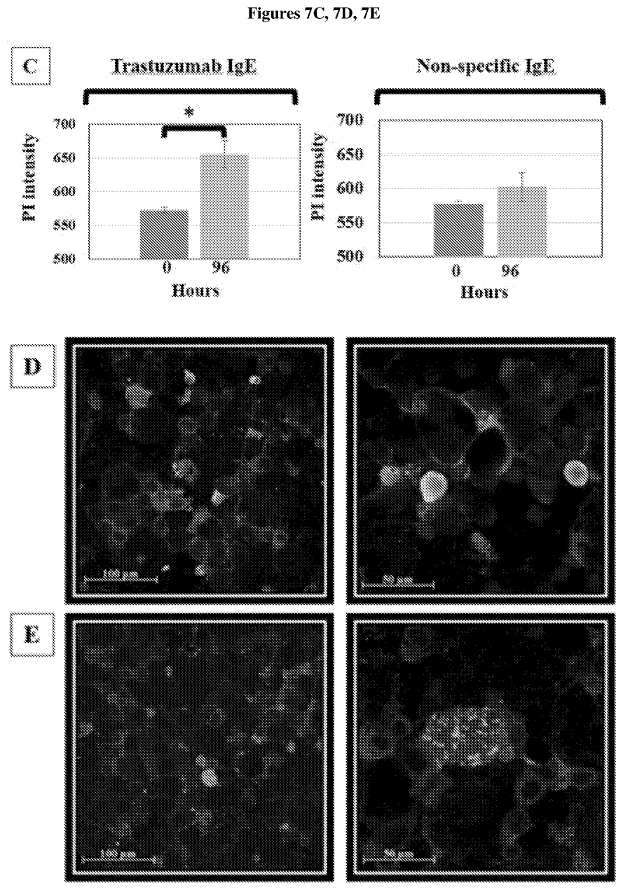

The ability of ADMC to induce breast cancer cell death was investigated. The IgE anti-HER2/neu sensitized ADMC were added to SK-BR-3 cells in medium containing PI to discriminate dead cells from live cells [146]. As seen in FIG. 7A, binding of anti-HER2/neu-sensitized (trastuzumab IgE) ADMC to SK-BR-3 cells induced significant cell death of the breast cancer cells as assessed by the uptake and visualization (red) of the PI in the SK-BR-3 cells but not the ADMC. The ADMC sensitized with non-specific IgE did not significantly affect the viability of SK-BR-3 cells (FIG. 7B). Quantification of the PI signal in FIG. 7C demonstrated significant (p=0.0003) breast cancer cell killing using trastuzumab IgE sensitization of the ADMC. Similarly, anti-HER2/neu IgE C6MH3-B1 sensitized ADMC induced significant (p=0.032) SK-BR-3 cell death (data not shown). No significant breast cancer cell death was observed when ADMC were sensitized with psIgE. In addition, ADMC added to the SK-BR-3 over 72 hours revealed breast cancer cell death, but not anti-tryptase labelled ADMC death, as indicated by immunostaining SK-BR-3 with an antibody specific for the apoptotic enzyme caspase 3 (FIG. 7D and FIG. 7E). These experiments indicate ADMC binding to SK-BR-3 results in FcϵRI activation and ADMC degranuation capable of inducing SK-BR-3 cell death.

IV.D. Mediators Released from ADMC Through FcϵRI Induce SK-BR-3 Cell Death

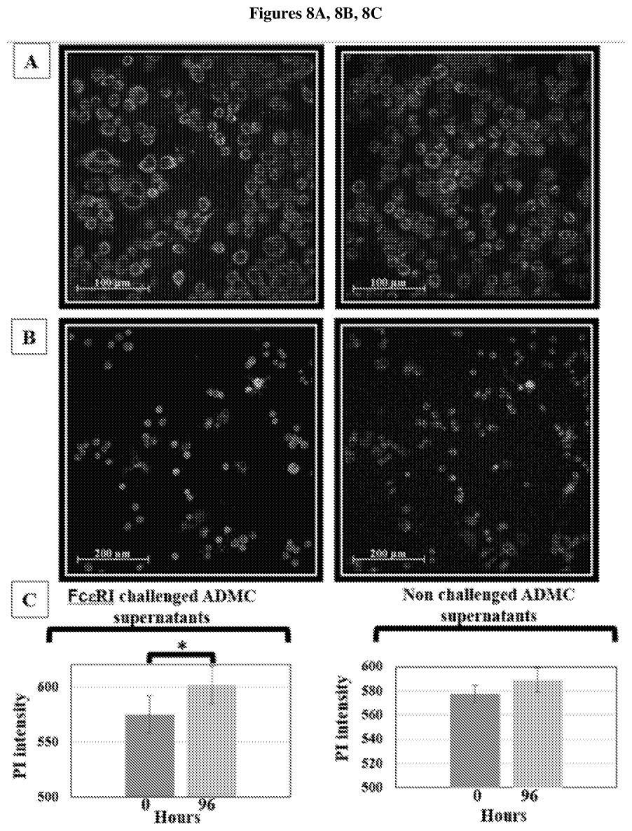

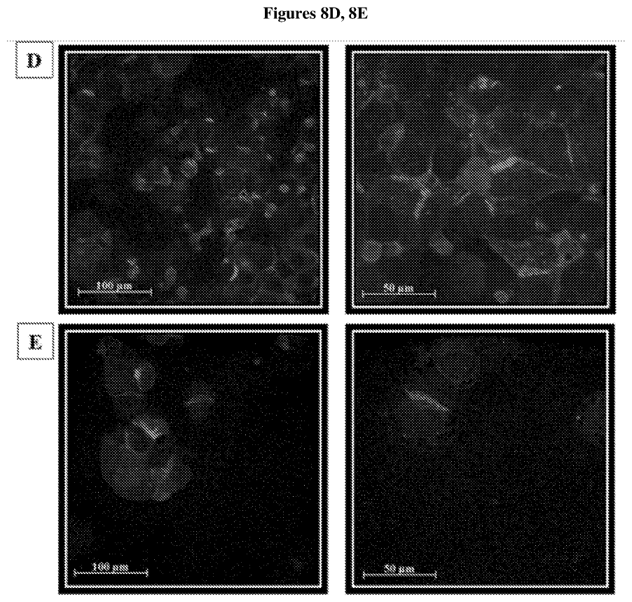

As shown above, ADMC produce anti-tumor mediators upon FcϵRI activation following cross-linking. The ability of mediators obtained from FcϵRI-activated ADMC to induce SK-BR-3 cell death was examined As seen in FIG. 8A and FIG. 8B), media (no ADMC) from optimally activated ADMC (60% mediator release) incubated with SK-BR-3 cells induced SK-BR-3 cell death as assessed using PI uptake. Quantification of PI uptake (FIG. 8C) demonstrated that SK-BR-3 cell death was significantly higher (p=0.009) when incubated for four days with supernatants from optimally activated ADMC cells compared to supernatants from non-FcϵRI challenged ADMC. Further, when the media from optimally activated FcϵRI ADMC were added to the SK-BR-3, the SK-BR-3 stained positive for caspase 3 (FIG. 8D and FIG. 8E) indicating cell death of the breast cancer cells as in FIG. 7.

V. Alternative Materials and Methods: Sensitized Human Adipose-Derived Mast Cells

V.A. Sensitized Human Adipose-Derived Mast Cells with anti-HER2/neu IgE and the Kinetics of Release in Response to HER2/neu Antigen.

Cellular Activation and Measurement of Mediator Release

All tissue to obtain mast cell through extensive digestion [101] is obtained under IRB approval with appropriate clinical information. In initial experiments mast cells sensitized with IgE HER2/neu (a gift from Manuel Pinochet [102]) are challenged with HER2 antigen (from Acro Biosystems, Newark, Del.; generally 0.001 μg/m1-20 μg/ml), ECD^{HER} (monomers and dimers), or D2F2/E2 murine mammary cells that express human HER2/neu (or the parental cell line D2F2 as a control; provided by Dr. Wei-Zen Wei (Wayne State University, Detroit, Mich.). A non-HER2/neu -specific IgE (NS IgE) with human constant regions produced in the same manner alongside the anti-HER2/neu IgE and anti-HER2/neu IgG1 (obtained from Genentech). IgE to DNP (JW8 clone) are used as a control in conjunction with DNP alone or DNP coated 5 uM beads (that mimic cells).

V.B. Human Adipose Derived Mast Cells, Sensitized with Anti-HER2/neu IgE, Can Immuno-Detect HER2/neu-Positive Breast Cancer Cells, Degranulate, and Kill the Tumor Cells In Vitro.

Adipose Derived Mast Cells and Breast Cancer Cell Co-Culture and Confocal Microscopy

The human breast cancer cell line SK-BR-3 is obtained from ATCC (American Type Culture Collection, Manassas, Va.) and cultured in RPMI 1640 (Invitrogen Corporation, Carlsbad, Calif.). D2F2/E2 murine mammary cells that express human HER2/neu and the parental cell line D2F2 (syngeneic to BALB/c mice) are obtained from Dr. Wei-Zen Wei (Wayne State University, Detroit, Mich.) and grown in IMDM medium (Invitrogen Corporation). Adipose-derived mast cells are obtained as described above. As a control human skin derived mast cells are used [88].

Mast cells sensitized with anti-HER2/neu IgE are cultured in dual chambered culture plates with HER2/neu expressing SK-BR-3. There are several specially made slide chambers and culture chambers available for these types of studies. While these are not pure “chemotactic” experiments (i.e. no concentration gradient for mast cell attraction would be observed) several commercially available products are available that have chemotactic characteristics with non flow and flow capabilities (Ibidi.com). While measuring cell binding under physiological flow conditions (as was done with human blood cells attaching to endothelium [104]) is not necessary for these experiments, the focus is on “flowing” the mast cells past the breast cancer cells which mimics what would happen in the circulatory system if mast cells were injected into the bloodstream. Given that the breast cancer cells are adherent, mast cells are continually pumped over them at different cells concentrations (generally 0.5×10⁶ to 1×10⁶ cells/ml) and flow rates (5 to 100 ml/minute), cells harvested and cytospins made (or culture slides used) for immunohistochemistry (see below) or confocal/live cell microscopy.

In the first set of experiments mast cell binding to SK-BR-3 cells are assessed by confocal microscopy [97, 105]. In these experiments cells are labelled with different color dyes so they can be distinguished in vitro. Mast cells are labeled with Hoerst and breast cancer cells labelled with mito-tracker, of course before adding to dual culture to avoid cross-labelling contamination. The dyes used here are not critical (there are many organelle-specific dyes available commercially) as long as the two cell types can be distinguished and dyes that allow for maximal identification of each cell type may be identified. Cells are added at different sides of the culture chamber and live cells followed over time using a Zeiss live cell imaging confocal microscopy as described in the facilities section. Cells are incubated over several hours and/or days and cell attachment assessed.

Adipose derived mast cells and breast cancer cell co-culture and apoptosis

In order to determine if the adipose derived mast cells can induce apoptosis in breast cancer cells, standard apoptosis assays as described previously are used [106-108]. Dye-labelled (e.g. endotracker green; different color from PE) breast cancer cells are stained using PE-Annexin V Apoptosis Detection Kit I (BD, USA) according to the manufacturer's protocol. Briefly, cells are washed with PBS and resuspended in binding buffer and PE-Annexin V followed by 7-AAD added to the cells. Propidium iodide can also be used here. The mixture is gently vortexed and incubated for 15 minutes at room temperature in the dark. Binding buffer is added to each tube and samples analyzed using dual labeling flow cytometry. Gating on dual-labelled breast cancer cells (e.g. organelle-specific green or blue labelled with red emitting PE-annexin) will distinguish between differentially labelled mast cells. Breast cancer cells will have mast cells bound to them and it may have to be determined if the mast cells must be freed from the breast cancer cells for FACs to be performed.

A second way in which apoptotic cells could be assessed is by immunohistochemistry of dual labelled cells. [97, 98, 100, 109-111]. In this protocol, cytospins of mast cells bound to breast cancer cells are fixed, blocked, and double labelled with mouse anti-tryptase (mast cells) and apotostis detecting antibodies (for which many exist e.g. active caspase-3, cytokeratin-18, cleaved lamin A, phosphorylated histone H2AX, cleaved poly(ADP ribose) polymerase) [112]. Extra special care ought to be taken to include proper controls due to the complexity of such a dual labelling—each primary must be from a different species and isotype controls used in parallel to each primary antibody as described in previous publications. Variously dyed, F(ab)₂ secondary antibodies are utilized so as to minimize cross reactivity with FcϵRI -expressing mast cells.

VII. Anti-Tumor Activity of TNF-α Containing Human Mast Cells In Vivo.

It is hypothesized that that human IgE anti-HER2/neu sensitized mast cells will detect HER2 positive cells in tumors, degranulate, induce tumor necrosis through TNF-a, and increase life span of mice.

A model used to test the anti-tumor activity of the fully human IgE anti-HER/neu antibody will be utilized first [89]. Briefly, female BALB/c human FcϵRIα transgenic mice 6-12 weeks old are challenged intraperitoneally (i.p.) with 2×˜10⁵ D2F2/E2 cells. Two days after tumor challenge, age matched mice are treated i.p. with 100 μg of the anti-HER2/neu IgE sensitized human mast cells or Hank's Balanced Salt Solution (HBSS) alone (5-6 animals per group per experiment). Mice are treated i.p. for a second time on day 4 with 100 μg of the same treatments and survival recorded. Kaplan-Meier plots are generated using GraphPad Prism 4. Statistical analysis is performed using this software and the Log Rank Test. Serum is saved for analysis of tryptase [126, 127] and TNF-α release [117].

The above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

### REFERENCES

1. Molderings, G. J., (2010). Mast Cell function in physiology and pathophysiology. Biotrend Rev., vol. 5, no. 1, 1-12.

2. Ribatti, D., (2016). The development of human mast cells. An historical reappraisal. Exp Cell Res, vol. 342, no. 2, 210-215.

3. Krystel-Whittemore, M , Dileepan, K. N., and J. G. Wood, (2016). Mast cell: A multi-functional master cell. Front Immunol, vol. 6, no. JAN, 1-12.

4. Zhang, B., Asadi, S., Weng, Z., Sismanopoulos, N., and T. C. Theoharides, (2012). Stimulated Human Mast Cells Secrete Mitochondrial Components That Have Autocrine and Paracrine Inflammatory Actions. PLoS One, vol. 7, 12.

5. Serafini, M. and C. M. Verfaillie, (2006) Pluripotency in adult stem cells: State of the art. Semin Reprod Med, vol. 24, 5.

6. Toma, J. G., Akhavan, M., Fernandes, K. J. L., Barnabé-Heider, F., Sadikot, A., Kaplan, D. R. and F. D. Miller, (2001) Isolation of multipotent adult stem cells from the dermis of mammalian skin. Nat Cell Biol, vol. 3, 9.

7. Goodell, M. A.,. Brose, K., Paradis, G., Conner, A. S. and R. C. Mulligan, (1996) Isolation and functional properties of murine hematopoietic stem cells that are replicating in vivo. J Exp Med, vol. 183, 4.

8. Messina, E., De Angelis, L., Frati, G., Morrone, S., Chimenti, S., Fiordaliso, F., Salio, M., Battaglia, M.,. Latronico, M. V. G., Coletta, M., Vivarelli, E., Frati, L., Cossu, G. and A. Giacomello, (2004), Isolation and expansion of adult cardiac stem cells from human and murine heart. Circ Res, vol. 95, 9.

9. Seo, B.-M., Miura, M., Gronthos, S., Bartold, P. M.,. Batouli, S., Brahim, J., Young, M., Robey, G., Wang, C.-Y and S. Shi, (2004). Investigation of multipotent postnatal stem cells from human periodontal ligament. Lancet, vol. 364, 9429.

10. Wang, -Y. and S. Shi, (2004) Investigation of multipotent postnatal stem cells from human periodontal ligament, Lancet, vol. 364, no. 9429.

11. Kern, S., Eichler, H., Stoeve, J., Klüter, H. and K. Bieback, (2006) Comparative analysis of mesenchymal stem cells from bone marrow, umbilical cord blood, or adipose tissue. Stem Cells, vol. 24, 5.

12. Branch, M .J., Yu, W.-Y., Sheridan, C. and A. Hopkinson, (2014). Isolation of adult stem cell populations from the human cornea, vol. 1235.

13. Baek, H., Noh, Y. H., Lee, J. H., Yeon, S. -I., Jeong, J. and H. Kwon, (2014). Autonomous isolation, long-term culture and differentiation potential of adult salivary gland-derived stem/progenitor cells. J Tissue Eng Regen Med, vol. 8, 9.

14. Gimble, J. M., and F. Guilak, (2003). Adipose-derived adult stem cells: Isolation, characterization, and differentiation potential. Cytotherapy, vol. 5, 5.

15. Hill, J. M., Zalos, G., Halcox, J. P. J., Schenke, W. H., Waclawiw, M. A., Quyyumi, A. A., and T. Finkel, (2003). Circulating endothelial progenitor cells, vascular function, and cardiovascular risk. N Engl J Med, vol. 348, 7.

16. Trosko J. E. and C.-C. Chang, (2002). Isolation and Characterization of Normal Adult Human Epithelial Pluripotent Stem Cells. Oncol Res, vol. 13, 6-10.

17. Nguyen, A., Guo, J., Banyard, D. A., Fadavi, D., Toranto, J. D., Wirth, G. A., Paydar, K. Z., Evans, G. R. D. and A. D. Widgerow, (2016). Stromal vascular fraction: A regenerative reality? Part 1: Current concepts and review of the literature. J Mast Reconstr Aesthetic Surg, vol. 69, no. 2, 170-179.

18. Bourin, P., Bunnell, B. A., Casteilla, L., Dominici, M., Katz, A. J., March, K. L., Redl, H., Rubin, J. P., Yoshimura, K. and J. M. Gimble, (2013). Stromal cells from the adipose tissue-derived stromal vascular fraction and culture expanded adipose tissue-derived stromal/ stem cells: a joint statement of the International Federation for Adipose Therapeutics (IFATS) and Science and the International S. Cytotherapy, vol. 15, no. 6, 641-648.

19. Gimble, J. M.,. Bunnell, B. A.,. Chiu, E. S. and F. Guilak, (2011) Concise review: Adipose-derived stromal vascular fraction cells and stem cells: Let's not get lost in translation. Stem Cells, vol. 29, no. 5, 749-754.

20. Riordan, N. H., Ichim, T E , Min, W., -P., Wang, H., Solano, F., Lara, F., Alfaro, M., Rodriguez, J. P., Harman, R. J., Patel, A. N., Murphy, M. P., Lee, R. R. and B. Minev, (2009) Non-expanded adipose stromal vascular fraction cell therapy for multiple sclerosis. J Transl Med, vol. 7, 29.

21. Irani, A.-M. A., Huang, C., Xia, H. -Z., Kepley, C., Nafie, A., El Fouda, S. Craig, B. Zweiman, and L. B. Schwartz, (1998) Immunohistochemical detection of human basophils in late-phase skin reactions. J Allergy Clin Immunol, vol. 101, 3.

22. Norton, S. K., Dellinger, A., Zhou, Z., Lenk, R., MacFarland, D., Vonakis, B., Conrad, D. and C. L. Kepley, (2010). A new class of human mast cell and peripheral blood basophil stabilizers that differentially control allergic mediator release. Clin Transl Sci, vol. 3, no. 4, 158-169.

23. Brooks, B., Norton, S., Dellinger, A., Zhou, Z., Lenk, R., Land, D. M.-, Conrad, D. and C. L. Kepley, (2013). Effects of Novel Nanomaterials on Allergic Mediator Release from Human Mast cells through Non-IgE Mediated Pathways. J Allergy Clin Immunol, vol. 123, no. 2, S25.

24. Kovarova, M., Latour, A. M., Chason, K. D., Tilley, S. L. and B. H. Koller, (2010). Human embryonic stem cells : a source of mast cells for the study of allergic and inflammatory diseases. Differentiation, vol. 115, no. 18, 3695-3703.

25. Kinoshita, T., Sawai, N., Hidaka, E., Yamashita, T. and K. Koike, (1999). Interleukin-6 directly modulates stem cell factor-dependent development of human mast cells derived from CD34+ cord blood cells. Blood, vol. 94, 2.

26. Furitsu, T., Saito, H., Dvorak, A. M., Schwartz, L. B., Irani, A.-M. A., Burdick, J. F., Ishizaka, K. and T. Ishizaka, (1989). Development of human mast cells in vitro. Proceedings of the National Academy of Sciences of the United States of America, vol. 86, 24.

27. Irani, A. A., Craig, S. S., Nilsson, G., Ishizaka, T. and L. B. Schwartz, (1992). Characterization of human mast cells developed in vitro from fetal liver cells cocultured with murined 3T3 fibroblasts. Immunology, vol. 77, 136-143.

28. Irani, A.-M. A., Nilsson, G., Miettinen, U., Craig, S. S., Ashman, L. K., Ishizaka, T., Zsebo, K. M. and L. B. Schwartz, (1992). Recombinant human stem cell factor stimulates differentiation of mast cells from dispersed human fetal liver cells. Blood, vol. 80, 12.

29. Mitsui, H., Furitsu, T., Dvorak, A. M., Irani, A.-M. A.,. Schwartz, L. B., Inagaki, N., Takei, M., Ishizaka, K., Zsebo, K. M., Gillis, S. and T. Ishizaka, (1993). Development of human mast cells from umbilical cord blood cells by recombinant human and murine c-kit ligand, Proceedings of the National Academy of Sciences of the United States of America, vol. 90, 2.

30. Butterfield, J. H., Weiler, D., Dewald, G. and G. J. Gleich, (2003). Establishment of an immature mast cell line from a patient with mast cell leukemia. Leuk. Res., vol. 12, 4.

31. Kirshenbaum, A. S., Akin, C., Wu, Y., Rottem, M., Goff, J. P., Beaven, M. A., Rao,

V. K. and D. D. Metcalfe, (2003). Characterization of novel stem cell factor responsive human mast cell lines LAD 1 and 2 established from a patient with mast cell sarcoma/leukemia; Activation following aggregation of FcϵRI or FcγRI. Leuk. Res., vol. 27, 8.

32. Siebenhaar, F., Falcone, F. H., Tiligada, E., Hammel, I., Maurer, M.,. Sagi-Eisenberg, R. and F. Levi-Schaffer, (2015). The search for mast cell and basophil models—Are we getting closer to pathophysiological relevance? Allergy Eur. J. Allergy Clin. Immunol., vol. 70, no. 1, 1-5.

33. Kepley, C. L. and N. Cohen, (2003). Evidence for human mast cell nonreleaser phenotype. J. Allergy Clin. Immunol., vol. 112, 2.

34. Kepley, C. L., (2005). Antigen-induced reduction in mast cell and basophil functional responses due to reduced syk protein levels. Int. Arch. Allergy Immunol., vol. 138, no. 1, 29-39.

35. Kambe, N., Kambe, M., Kochan, J. P. and L. B. Schwartz, (2001). Human skin-derived mast cells can proliferate while retaining their characteristic functional and protease phenotypes. Blood, vol. 97, 2045-2052.

36. Kambe, N., Kambe, M., Chang, H.-W., Matsui, A., M M, H.-K., Hussein, M., Oskerizian, C. A., Kochan, J., Irani, A.-M. A. and L. B. Schwartz, (2000). An improved procedure for the development of human mast cells from dispersed fetal liver cells in serum-free culture medium. J. Immunol. Methods, vol. 240, 101-110.

37. Irani, A.-M. A., Bradford, T. R., Kepley, C. L.,. Schechter, N. M. and L. B. Schwartz, (1989). Detection of MC(T) and MC(TC) types of human mast cells by immunohistochemistry using new monoclonal anti-tryptase and anti-chymase antibodies. J. Histochem. Cytochem., vol. 37, no. 10.

38. Kepley, C. L., Pfeiffer, J. R., Schwartz, L. B., Wilson, B. S. and J. M. Oliver, (1998). The identification and characterization of umbilical cord blood-derived human basophils. J. Leukoc. Biol., vol. 64, no. 4.

39. Ogawa, M., Nakahata, T., Leary, A. G., Sterk, A. R., Ishizaka, K. and T. Ishizaka, (1983). Suspension culture of human mast cells/basophils from umbilical cord blood mononuclear cells. Proc. Natl. Acad. Sci. U.S.A., vol. 80, no. 14 I.

40. Saito, H., Ebisawa, M., Tachimoto, H.,. Shichijo, M., Fukagawa, K., Matsumoto, K., Iikura, Y., Awaji, T., Tsujimoto, G., Yanagida, M., Uzumaki, H., Takahashi, G., Tsuji, K. and T. Nakahata, (1996). Selective growth of human mast cells induced by steel factor, IL-6, and prostaglandin E2 from cord blood mononuclear cells. J. Immunol., vol. 157, 1.

41. Thomson, J. A., (1998) Embryonic stem cell lines derived from human blastocysts. Science (80-.)., vol. 282, no. 5391.

42. Woo, S. H., Byeong, C. L., Chang, K. L. and K. K. Sung, (2005). Human embryonic stem cells and therapeutic cloning, J. Vet. Sci., vol. 6, no. 2.

43. Denning C. and H. Priddle, (2003). New frontiers in gene targeting and cloning: Success, application and challenges in domestic animals and human embryonic stem cells. Reproduction, vol. 126, no. 1.

44. Stojkovic, M., Lako, M., Strachan, T. and A. Murdoch, (2004). Derivation, growth and applications of human embryonic stem cells. Reproduction, vol. 128, no. 3.

45. Slukvin, I. I.,. Vodyanik, M. A., Thomson, J. A., Gumenyuk, M. E. and E.-D. Choi, (2006). Directed differentiation of human embryonic stem cells into functional dendritic cells through the myeloid pathway. J. Immunol., vol. 176, no. 5.

46. Zhan, X., Dravid, G., Ye, Z., Hammond, H., Shamblott, M., Gearhart, J.and L. Cheng, (2004) Functional antigen-presenting leucocytes derived from human embryonic stem cells in vitro. Lancet, vol. 364, no. 9429.

47. Ehrlich, P., (1877). Beitrage Zur Kenntis der Anilinfarbugen and ihrer Verwendung in der Mikroskepischen Technik.pdf,.

48. Maltby, S., Khazaie, K. and K. M. McNagny, (2009). Mast cells in tumor growth: angiogenesis, tissue remodelling and immune-modulation. Biochim. Biophys. Acta, vol. 1796, no. 1, 19-26.

49. Bagó, J. R., Alfonso-Pecchio, A., Okolie, O., Dumitru, R., Rinkenbaugh, A., Baldwin, A. S., Miller, C. R, Magness, S. T. and S. D. Hingtgen, (2016). Therapeutically engineered induced neural stem cells are tumour-homing and inhibit progression of glioblastoma. Nat. Commun., vol. 7, 10593.

50. Schwartz, L. B., and Huff, T. F., (1993). Biology of mast cells and basophils. In Allergy: Principals and Practice, Volume 4, E. Middleton, Jr., C. E. Reed, E. F. Ellis, N. F. Adkinson, Jr., J. W. Yunginger and W. W. Busse, eds. (St. Louis: Mosby-Year Book, Inc.), pp. 135-168.

51. Mukai, K., Tsai, M, Saito H, and S. J. Galli, (2018). Mast cells as sources of cytokines, chemokines, and growth factors. Immunol Rev 282:121-50.

52. Epstein, M. M., (2004). Do mouse models of allergic asthma mimic clinical disease? International Archives of Allergy and Immunology 133:84-100.

53. Finkelman, F. D., (2007). Anaphylaxis: lessons from mouse models. J. Allergy Clin. Immunol. 120:506-15

54. Lee, E., Min, H. K., Oskeritzian, C. A., Kambe, N., Schwartz, L. B., and C. H. Wook,. (2003). Recombinant human (rh) stem cell factor and rhIL-4 stimulate differentiation and proliferation of CD3+ cells from umbilical cord blood and CD3+ cells enhance FcepsilonRI expression on fetal liver-derived mast cells in the presence of rhIL-4. Cell Immunol, vol. 226, 30-36.

55. Nilsson, G., Blom, T., Kusche-Gullberg, M., Kjellen, L., Butterfield, J. H., Sundstrom, C., Nilsson, K., and L. Hellman, (1994). Phenotypic characterization of the human mast-cell line HMC-1. Scandinavian Journal of Immunology, vol. 39, 489-498.

56. Kirshenbaum, A. S., Akin, C., Wu, Y., Rottem, M., Goff, J. P., Beaven, M. A., Rao, V. K., and D. D. Metcalfe, (2003). Characterization of novel stem cell factor responsive human mast cell lines LAD 1 and 2 established from a patient with mast cell sarcoma/leukemia; activation following aggregation of FcepsilonRI or FcgammaRI. Leuk.Res., vol. 27, 677-682.

57. Dvorak, A. M., and E. S. Morgan, (1996). Ultrastructural detection of histamine in human mast cells developing from cord blood cells cultured with human or murine recombinant c-kit ligands. Int Arch Allergy Immunol, vol. 111, 238-244.

58. Hwang, W. S., Lee, B. C., Lee, C. K., and S. K. Kang, (2005). Human embryonic stem cells and therapeutic cloning. J Vet. Sci., vol. 6, 87-96.

59. Thomson, J. A., Itskovitz-Eldor, J., Shapiro, S. S., Waknitz, M. A., Swiergiel, J. J., Marshall, V. S., and J. M. Jones, (1998). Embryonic stem cell lines derived from human blastocysts. Science, vol. 282, 1145-1147.

60. Kovarova, M., Latour, A. M., Chason, K. D., Tilley, S. L., and B. H. Koller, (2010). Human embryonic stem cells: a source of mast cells for the study of allergic and inflammatory diseases. Blood, vol. 115, 3695-3703.

61. Dabiri, S., Huntsman, D., Makretsov, N., Cheang, M., Gilks, B., Bajdik, C., Gelmon, K., Chia, S., and M. Hayes, (2004). The presence of stromal mast cells identifies a subset of invasive breast cancers with a favorable prognosis. Mod Pathol, vol. 17, 690-695.

62. Zhao, W., Oskeritzian, C. A., Pozez, A. L., and L. B. Schwartz, (2005). Cytokine production by skin-derived mast cells: endogenous proteases are responsible for degradation of cytokines. J Immunol. 175:2635-42.

63. Gomez, G., Schwartz, L., and Kepley, C., (2007). Syk deficiency in human non-releaser lung mast cells. Clin Immunol, vol. 125, 112-115.

64. Saudemont, A., Jespers, L., and T. Clay, (2018). Current Status of Gene Engineering Cell Therapeutics. Front Immunol 9:153.

65. Josiah, D. T., Zhu, D., Dreher, F., Olson, J., McFadden, G., and H. Caldas, (2009). Adipose-derived Stem Cells as Therapeutic Delivery Vehicles of an Oncolytic Virus for Glioblastoma. Mol Ther, vol 18, 377-385.

66. Zhao, W., Gomez, G., Macey, M., Kepley, C. L., and L. B. Schwartz, (2011). In Vitro Desensitization of Human Skin Mast Cells. J Clin Immunol., vol. 32, 150-160.

67. Imai, T., Koike, K., Kubo, T., Kikuchi, T., Amano, Y., Takagi, M., Okumura, N., and T. Nakahata, (1991). Interleukin-6 supports human megakaryocytic proliferation and differentiation in vitro. Blood, vol. 78, 1969-1974.

68. de Vries V. C., Wasiuk A., Bennett, K. A., Benson, M. J., Elgueta, R., and T. J. Waldschmidt et al. (2009) Mast cell degranulation breaks peripheral tolerance. Am J Transplant 9:2270-80.

69. Karagiannis, S. N., Josephs, D. H., Bax, H. J., and J. F. Spicer, (2017). Therapeutic IgE Antibodies: Harnessing a Macrophage-Mediated Immune Surveillance Mechanism against Cancer. Cancer Res 77:2779-83.

70. Daniels, T. R., Martinez-Maza, O., and M. L. Penichet, (2012). Animal models for IgE-meditated cancer immunotherapy. Cancer Immunol Immunother 61, 1535-1546.

71. Leoh, L. S., Daniels-Wells, T. R., and M. L. Penichet, (2015). IgE immunotherapy against cancer. Current topics in microbiology and immunology 388, 109-149.

72. Matthews, N., and J. F. Watkins, (1978). Tumour-necrosis factor from the rabbit. I. Mode of action, specificity and physicochemical properties. British journal of cancer 38, 302-309.

73. Green, S., Dobrjansky, A., and M. A. Chiasson, (1982). Murine tumor necrosis-inducing factor: purification and effects on myelomonocytic leukemia cells. J Natl Cancer Inst 68, 997-1003.

74. Wang, X., and Y. Lin, (2008). Tumor necrosis factor and cancer, buddies or foes? Acta Pharmacol Sin 29, 1275-1288.

75. Wang, X., Chen, W., and Y. Lin, (2007). Sensitization of TNF-induced cytotoxicity in lung cancer cells by concurrent suppression of the NF-kappaB and Akt pathways. Biochem Biophys Res Commun 355, 807-812.

76. Hayes, A. J., Neuhaus, S. J., Clark, M. A., and J. M. Thomas, (2007). Isolated limb perfusion with melphalan and tumor necrosis factor alpha for advanced melanoma and soft-tissue sarcoma. Ann Surg Oncol 14, 230-238.

77. Grunhagen, D. J., de Wilt, J. H., ten Hagen, T. L., and A. M. Eggermont, (2006). Technology insight: Utility of TNF-alpha-based isolated limb perfusion to avoid amputation of irresectable tumors of the extremities. Nat Clin Pract Oncol 3, 94-103.

78. Lans, T. E., Grunhagen, D. J., de Wilt, J. H., van Geel, A. N., and A. M. Eggermont, (2005). Isolated limb perfusions with tumor necrosis factor and melphalan for locally recurrent soft tissue sarcoma in previously irradiated limbs. Ann Surg Oncol 12, 406-411.

79. Rossi, C. R., Mocellin, S., Pilati, P., Foletto, M., Campana, L., Quintieri, L., De Salvo, G. L., and M. Lise, (2005). Hyperthermic isolated perfusion with low-dose tumor necrosis factor alpha and doxorubicin for the treatment of limb-threatening soft tissue sarcomas. Ann Surg Oncol 12, 398-405.

80. Di Filippo, F., Garinei, R., Anza, M., Cavaliere, F., Giannarelli, D., Cagol, P. P., Rossi, C. R., Santinami, M., Deraco, M., and C. Botti, et al. (2003). Doxorubicin in isolation limb perfusion in the treatment of advanced limb soft tissue sarcoma. J Exp Clin Cancer Res 22, 81-87.

81. van Etten, B., de Vries, M. R., van, I. M. G., Lans, T. E., Guetens, G., Ambagtsheer, G., van Tiel, S. T., de Boeck, G., de Bruijn, E. A., and A. M. Eggermont, et al. (2003). Degree of tumour vascularity correlates with drug accumulation and tumour response upon TNF-alpha-based isolated hepatic perfusion. British journal of cancer 88,314-319.

82. Oshiro, S., Tsugu, H., Komatsu, F., Ohnishi, H., Ueno, Y., Sakamoto, S., Fukushima, T., and G. Soma, (2006). Evaluation of intratumoral administration of tumor necrosis factor-alpha in patients with malignant glioma. Anticancer Res 26, 4027-4032.

83. Cornett, W. R., McCall, L. M., Petersen, R. P., Ross, M. I., Bridle, H. A., Noyes, R. D., Sussman, J. J., Kraybill, W. G., Kane, J. M., 3rd, and H. R. Alexander, et al. (2006). Randomized multicenter trial of hyperthermic isolated limb perfusion with melphalan alone compared with melphalan plus tumor necrosis factor: American College of Surgeons Oncology Group Trial Z0020. Journal of clinical oncology: official journal of the American Society of Clinical Oncology 24, 4196-4201.

84. Chiavenna, S. M., Jaworski, J. P., and A. Vendrell, (2017). State of the art in anti-cancer mAbs. J Biomed Sci 24, 15.

85. Vogel, C. L., Cobleigh, M. A., Tripathy, D., Gutheil, J. C., Harris, L. N., Fehrenbacher, L., Slamon, D. J., Murphy, M., Novotny, W. F., and M. Burchmore, et al. (2001). First-line Herceptin monotherapy in metastatic breast cancer. Oncology 61 Suppl 2, 37-42.

86. Vogel, C., Cobleigh, M. A., Tripathy, D., Gutheil, J. C., Harris, L. N., Fehrenbacher, L., Slamon, D. J., Murphy, M., Novotny, W. F., and M. Burchmore, et al. (2001). First-line, single-agent Herceptin(R) (trastuzumab) in metastatic breast cancer. a preliminary report. Eur J Cancer 37 Suppl 1, 25-29.

87. Kepley, C. L., Taghavi, S., Mackay, G., Zhu, D., Morel, P. A., Zhang, K., Ryan, J. J., Satin, L. S., Zhang, M., and P. P. Pandolfi, et al. (2004). Co-aggregation of FcgammaRII with FcepsilonRI on human mast cells inhibits antigen-induced secretion and involves SHIP-Grb2-Dok complexes. J Biol Chem 279, 35139-35149.

88. Daniels, T. R., Leuchter, R. K., Quintero, R., Helguera, G., Rodriguez, J. A., Martinez-Maza, O., Schultes, B. C., Nicodemus, C. F., and M. L. Penichet, (2012). Targeting HER2/neu with a fully human IgE to harness the allergic reaction against cancer cells. Cancer Immunol Immunother 61, 991-1003.

89. Mestas J, and C. C. Hughes, (2004) Of mice and not men: differences between mouse and human immunology. J Immunol 172:2731-8.

90. Leoh, L. S., Daniels-Wells, T. R., and M. L. Penichet, (2015). IgE immunotherapy against cancer. Curr Top Microbiol Immunol 388:109-49.

91. Ribatti, D., and E. Crivellato, (2011). Mast cells, angiogenesis and cancer. Advances in experimental medicine and biology 716, 270-288.

92. Kinet, J. P., (1999). The high-affinity IgE receptor (Fc epsilon RI): from physiology to pathology. Annu.Rev.Immunol. 17:931-72.:931-72.

93. Sinnamon, M. J., Carter, K. J., Sims, L. P., Lafleur, B., Fingleton, B., and L. M. Matrisian, (2008). A protective role of mast cells in intestinal tumorigenesis. Carcinogenesis 29, 880-886.

94. Johansson, A., Rudolfsson, S., Hammarsten, P., Halin, S., Pietras, K., Jones, J., Stattin, P., Egevad, L., Granfors, T., and P. Wikstrom, et al. (2010). Mast cells are novel independent prognostic markers in prostate cancer and represent a target for therapy. The American Journal of Pathology 177, 1031-1041.

95. Welsh, T. J., Green, R. H., Richardson, D., Waller, D. A., O'Byrne, K. J., and P. Bradding, (2005). Macrophage and mast-cell invasion of tumor cell islets confers a marked survival advantage in non-small-cell lung cancer. Journal of clinical oncology: official journal of the American Society of Clinical Oncology 23, 8959-8967.

96. Dellinger, Anthony, Brooks, D. B., Plunkett, B., Vonakis, B. M., Sandros, M. et al. and Christopher L. Kepley * (2012). Effects of Novel Nanomaterials on Allergic Mediator Release from Human Mast Cells and Basophils through Non-Ige Mediated Pathways. J Nanomed Nanotechol 3, 8.

97. Dellinger, A., Zhou, Z., Norton, S. K., Lenk, R., Conrad, D., and C. L. Kepley, (2010). Uptake and distribution of fullerenes in human mast cells. Nanomedicine 6, 575-582.

98. Jensen-Jarolim, E., Achatz, G., Turner, M. C., Karagiannis, S., Legrand, F., and Capron M, et al., (2008). AllergoOncology: the role of IgE-mediated allergy in cancer. Allergy 63:1255-66.

99. Kepley, C. L., Cambier, J. C., Morel, P. A., Lujan, D., Ortega, E., Wilson, B. S., and J. M. Oliver, (2000). Negative regulation of FcepsilonRI signaling by FcgammaRII costimulation in human blood basophils. J Allergy Clin Immunol 106, 337-348.

100. Almagro, J. C., Daniels-Wells, T. R., Perez-Tapia, S. M., and M. L. Penichet, (2017). Progress and Challenges in the Design and Clinical Development of Antibodies for Cancer Therapy. Front Immunol 8:1751.

101. Teo, P. Z., Utz, P. J., and J. A. Mollick, (2012). Using the allergic immune system to target cancer: activity of IgE antibodies specific for human CD20 and MUC1. Cancer Immunol Immunother 61:2295-309.

102. Karagiannis, P., Singer, J., Hunt, J., Gan, S. K., Rudman, S. M., Mechtcheriakova, D., Knittelfelder, R., Daniels, T. R., Hobson, P. S., and A. J. Beavil, et al. (2009). Characterisation of an engineered trastuzumab IgE antibody and effector cell mechanisms targeting HER2/neu-positive tumour cells. Cancer Immunol Immunother 58, 915-930.

103. Ferguson, K. M., Darling, P. J., Mohan, M. J., Macatee, T. L., and M. A. Lemmon, (2000). Extracellular domains drive homo- but not hetero-dimerization of erbB receptors. EMBO J 19, 4632-4643.

104. Kepley, C. L., Andrews, R. P., Brown, D. C., Chigaev, A., Sklar, L. A., Oliver, J. M., and R. S. Larson, (2002). Regulation of human basophil adhesion to endothelium under flow conditions: Different very late antigen 4 regulation on umbilical cord blood-derived and peripheral blood basophils. J Allergy Clin Immunol 110, 469-475.

105. Kepley, C. L., Wilson, B. S., and J. M. Oliver, (1998). Identification of the Fc epsilonRI-activated tyrosine kinases Lyn, Syk, and Zap-70 in human basophils. J Allergy Clin Immunol 102, 304-315.

106. Wang, J. X., Kaieda, S., Ameri, S., Fishgal, N., Dwyer, D., Dellinger, A., Kepley, C. L., Gurish, M. F., and P. A. Nigrovic, (2014). IL-33/ST2 axis promotes mast cell survival via BCLXL. Proceedings of the National Academy of Sciences of the United States of America 111, 10281-10286.

107. Norozian, F., Kashyap, M., Ramirez, C. D., Patel, N., Kepley, C. L., Barnstein, B. O., and J. J. Ryan, (2006). TGFbetal induces mast cell apoptosis. Exp Hematol 34, 579-587.

108. Wright, H. V., Bailey, D., Kashyap, M., Kepley, C. L., Drutskaya, M. S., Nedospasov, S. A., and J. J. Ryan, (2006). IL-3-mediated TNF production is necessary for mast cell development. J Immunol 176, 2114-2121.

109. Schwartz, L. B., and C. Kepley, (1994). Development of markers for human

110. Kepley, C. L., Youseff, L., Andrews, R. P., Wilson, B. S., and J. M. Oliver, (1999). Syk deficiency in non-releaser basophils. J. Allergy Clin Immunol 104, 279-284.

111. Irani, A. M., Huang, C., Xia, H. Z., Kepley, C., Nafie, A., Fouda, E. D., Craig, S., Zweiman, B., and L. B. Schwartz, (1998) Immunohistochemical detection of human basophils in late-phase skin reactions. J Allergy Clin Immunol 101, 354-362.

112. Holubec, H., Payne, C. M., Bernstein, H., Dvorakova, K., Bernstein, C., Waltmire, C. N., Warneke, J. A., and H. Garewal, (2005). Assessment of apoptosis by immunohistochemical markers compared to cellular morphology in ex vivo-stressed colonic mucosa. J Histochem Cytochem 53, 229-235.

113. See, D., Mason, S., and R. Roshan, (2002). Increased tumor necrosis factor alpha (TNF-alpha) and natural killer cell (NK) function using an integrative approach in late stage cancers. Immunological investigations 31, 137-153.

114. Nalbant, S., Akmaz, I., Kaplan, M., Avsar, K., Solmazgul, E., and B. Sahan, (2006). Does rofecoxib increase TNF-alpha levels? Clinical and experimental rheumatology 24, 361-365.

115. Wang, S., Yan, L., Wesley, R. A., and R. L. Danner, (1997). Nitric oxide increases tumor necrosis factor production in differentiated U937 cells by decreasing cyclic AMP.J Biol Chem 272, 5959-5965.

116. Borriello, F., Iannone, R., Di Somma, S., Loffredo, S., Scamardella, E., Galdiero, M. R., Varricchi, G., Granata, F., Portella, G., and G. Marone, (2016). GM-CSF and IL-3 Modulate Human Monocyte TNF-alpha Production and Renewal in In Vitro Models of Trained Immunity. Front Immunol 7, 680.

117. Dellinger, A. L., Cunin, P., Lee, D., Kung, A. L., Brooks, D. B., Zhou, Z., Nigrovic, P. A., and C. L. Kepley, (2015). Inhibition of inflammatory arthritis using fullerene nanomaterials. PloS One 10, e0126290.

118. Norton, S. K., Dellinger, A., Zhou, Z., Lenk, R., Macfarland, D., Vonakis, B., Conrad, D., and C. L. Kepley, (2010). A new class of human mast cell and peripheral blood basophil stabilizers that differentially control allergic mediator release. Clin Transl Sci 3, 158-169.

119. Norton, S. K., Wijesinghe, D. S., Dellinger, A., Sturgill, J., Zhou, Z., Barbour, S., Chalfant, C., Conrad, D. H., and C. L. Kepley, (2012). Epoxyeicosatrienoic acids are involved in the C(70) fullerene derivative-induced control of allergic asthma. J Allergy Clin Immunol 130, 761-769 e762.

120. Howe, L. R., and P. H. Brown, (2011). Targeting the HER/EGFR/ErbB family to prevent breast cancer. Cancer Prey Res (Phila) 4, 1149-1157.

121. Gould, H. J., Mackay, G. A., Karagiannis, S. N., O'Toole, C. M., Marsh, P. J., Daniel, B. E., Coney, L. R., Zurawski, V. R., Jr., Joseph, M., and M. Capron, et al., (1999). Comparison of IgE and IgG antibody-dependent cytotoxicity in vitro and in a SCID mouse xenograft model of ovarian carcinoma. European journal of immunology 29, 3527-3537.

122. Dellinger, A., Olson, J., Zhou, Z., Link, K., Vance, S., Sandros, M. G., Yang, J., Zhou, Z., and C. L. Kepley, (2013). Functionalization of gadolinium metallofullerenes for detecting atherosclerotic plaque lesions by cardiovascular magnetic resonance. Journal of cardiovascular magnetic resonance : official journal of the Society for Cardiovascular Magnetic Resonance 15, 7.

123. Xia, H. Z., Kepley, C. L., Sakai, K., Chelliah, J., Irani, A. M., and L. B. Schwartz, (1995). Quantitation of tryptase, chymase, Fc epsilon RI alpha, and Fc epsilon RI gamma mRNAs in human mast cells and basophils by competitive reverse transcription-polymerase chain reaction. J Immunol 154, 5472-5480.

124. Irani, A. A., Schechter, N. M., Craig, S. S., DeBlois, G., and L. B. Schwartz, (1986).

Two types of human mast cells that have distinct neutral protease compositions. Proceedings of the National Academy of Sciences of the United States of America 83, 4464-4468.

125. Irani, A. A., and L. B. Schwartz, (1989). Mast cell heterogeneity. Clinical and Experimental Allergy 19, 143-155.

126. Schwartz, L. B. (1990). Tryptase, a mediator of human mast cells. J Allergy Clin Immunol 86 Suppl., 594-598.

127. Wenzel, S., Irani, A. M., Sanders, J. M., Bradford, T. R., and L. B. Schwartz, (1986). Immunoassay of tryptase from human mast cells. J Immunol Methods 86, 139-142.

128. Rodewald, H. R. and T. B. Feyerabend, (2012). Widespread immunological functions of mast cells: fact or fiction? Immunity 37:13-24.

129. Passante, E., (2014) Mast cell and basophil cell lines: a compendium. Methods Mol Biol 1192:101-13.

130. Bischoff, S. C., (2007) Role of mast cells in allergic and non-allergic immune responses: comparison of human and murine data. Nat Rev Immunol 7:93-104.

131. Kirshenbaum, A. S., Kessler, S. W., Goff J. P., and D. D. Metcalfe, (1991). Demonstration of the origin of human mast cells from CD34+ bone marrow progenitor cells. Journal of Immunology 146:1410-5.

132. Jensen, B. M., Swindle, E. J., Iwaki, S., and A. M. Gilfillan, (2006). Generation, isolation, and maintenance of rodent mast cells and mast cell lines. Curr Protoc Immunol Chapter 3:Unit 3 23.

133. Varricchi, G., Galdiero, M. R., Loffredo, S., Marone, G , Iannone, R., and G. Marone, et al., (2017) Are Mast Cells MASTers in Cancer? Front Immunol 8:424.

134. Ribatti, D., (2016) Mast cells in lymphomas. Crit Rev Oncol Hematol 101:207-12.

135. Aaltomaa, S., Lipponen, P., Papinaho, S., and V. M. Kosma, (1993) Mast cells in breast cancer. Anticancer Res. 13:785-8.

136. Dalton, D. K., and R. J. Noelle, (2012) The roles of mast cells in anticancer immunity. Cancer Immunol Immunother 61:1511-20.

137. Varricchi, G., Galdiero, M. R., Marone, G., Granata, F., Borriello, F., and G. Marone, (2017) Controversial role of mast cells in skin cancers. Exp Dermatol 26:11-7.

138. Wasiuk, A., de Vries, V. C., Nowak, E. C., and R. J. Noelle, (2010). Mast Cells in Allergy and Tumor Disease. In “Cancer and IgE: Introducing the Concept of AllergoOncology”. Penichet M. L. and Jensen-Jarolim E., eds. Springer, New York, USA. pp. 137-158.

139. Yan, W. L., Shen, K. Y., Tien, C. Y., Chen, Y. A., and S. J. Liu, (2017). Recent progress in GM-CSF-based cancer immunotherapy. Immunotherapy 9:347-60.

140. Roberts, N. J., Zhou, S., Diaz, L. A., Jr., and M. Holdhoff, (2011). Systemic use of tumor necrosis factor alpha as an anticancer agent. Oncotarget 2:739-51.

141. Feisst, V., Meidinger, S., and M. B. Locke, (2015). From bench to bedside: use of human adipose-derived stem cells. Stem Cells Cloning 8:149-62.

142. Poglio, S., De Toni-Costes, F., Arnaud, E., Laharrague, P., Espinosa, E. and L. Casteilla et al., (2010) Adipose tissue as a dedicated reservoir of functional mast cell progenitors. Stem Cells 28:2065-72.

143. Ortiz-Sanchez, E., Helguera, G., Daniels, T. R., and M. L. Penichet, (2008).

Antibody-cytokine fusion proteins: applications in cancer therapy. Expert Opin Biol Ther 8:609-32.

144. Nfuritorton, A., Zhou, Z., Lenk, R., MacFarland, D., and C. L. Kepley, (2009). Fullerene nanomaterials inhibit phorbol myristate acetate-induced inflammation. Exp Dermatol 18:1079-81.

145. Dela Cruz, J. S., Lau, S. Y., Ramirez, E. M., De Giovanni. C., Forni. G, and S. L. Morrison et al., (2003). Protein vaccination with the HER2/neu extracellular domain plus anti-HER2/neu antibody-cytokine fusion proteins induces a protective anti-HER2/neu immune response in mice. Vaccine 21:1317-26.

146. Sasaki, D. T., Dumas, S. E., and E. G. Engleman, (1987). Discrimination of viable and non-viable cells using propidium iodide in two color immunofluorescence. Cytometry 8:413-20.

147. Gomez, G., Jogie-Brahim, S., Shima, M., and L. B. Schwartz, (2007). Omalizumab reverses the phenotypic and functional effects of IgE-enhanced Fc epsilonRI on human skin mast cells. J Immunol. 179:1353-61.

148. Lennon, S., Barton, C., Banken, L., Gianni, L., Marty, M., and J. Baselga, et al., (2009). Utility of serum HER2 extracellular domain assessment in clinical decision making: pooled analysis of four trials of trastuzumab in metastatic breast cancer. J Clin Oncol 27:1685-93.

149. Tse, C., Gauchez, A. S., Jacot, W., and P. J. Lamy, (2012). HER2 shedding and serum HER2 extracellular domain: biology and clinical utility in breast cancer. Cancer Treat Rev 38:133-42.

150. Ferguson, K. M., Darling, P. J., Mohan, M. J., Macatee, T. L., and M. A. Lemmon (2000). Extracellular domains drive homo- but not hetero-dimerization of erbB receptors. EMBO J 19:4632-43.

151. Kovacs, E., Zorn, J.A., Huang, Y., Barros, T., and J. Kuriyan, (2015). A structural perspective on the regulation of the epidermal growth factor receptor. Annu Rev Biochem 84:739-64.

152. Zschaler, J., Schlorke, D., and J. Arnhold, (2014). Differences in innate immune response between man and mouse. Crit Rev Immunol 34:433-54.

153. Galli, S. J., Tsai, M., and A. M. Piliponsky, (2008) The development of allergic inflammation. Nature 454:445-54.

154. Zhao, W., Kepley, C. L., Morel, P. A., Okumoto, L. M., Fukuoka, Y., and L. B. Schwartz, (2006). Fc gamma RIIa, not Fc gamma RIIb, is constitutively and functionally expressed on skin-derived human mast cells. J Immunol 177:694-701.

155. Furitsu, T., Arai, N., and T. Ishizaka, (1989). Co-culture of progenitors in human bone marrow (BM) and cord blood with human fibroblast for the differentiation of human mast cells. Fed.Proc. 3:A787.

156. Nilsson, G., Forsberg, K., Bodger, M. P., Ashman, L. K., Zsebo, K. M., and T. Ishizaka et al., (1993) Phenotypic characterization of stem cell factor-dependent human foetal liver-derived mast cells. Immunology 79:325-30.

157. Radinger, M., Jensen, B. M., Kuehn, H. S., Kirshenbaum, A., and A. M. Gilfillan, (2010). Generation, isolation, and maintenance of human mast cells and mast cell lines derived from peripheral blood or cord blood. Curr Protoc Immunol Chapter 7:Unit 7 37.

158. Kirshenbaum, A. S., Goff, J. P., Semere, T., Foster, B., Scott, L. M., and D. D. Metcalfe, (1999). Demonstration that human mast cells arise from a progenitor cell population that is CD34(+), c-kit(+), and expresses aminopeptidase N (CD13). Blood. 94:2333-42.

159. Maciel, T. T., Moura, I. C., and O. Hermine, (2015). The role of mast cells in cancers. F1000Prime Rep 7:09.

160. Wasiuk, A., de Vries, V. C., Hartmann, K., Roers, A., and R. J. Noelle, (2009). Mast cells as regulators of adaptive immunity to tumours. Clin Exp Immunol 155:140-6.

161. Pittoni, P., Tripodo, C., Piconese, S., Mauri, G., Parenza, M., and A. Rigoni et al., (2011). Mast cell targeting hampers prostate adenocarcinoma development but promotes the occurrence of highly malignant neuroendocrine cancers. Cancer Res 71:5987-97.

162. Gibbs, B. F., Wierecky, J., Welker, P., Henz, B. M., Wolff, H. H., and J. Grabbe, (2001). Human skin mast cells rapidly release preformed and newly generated TNF-alpha and IL-8 following stimulation with anti-IgE and other secretagogues. Exp.Dermatol. 10:312-20.

163. Gordon, J. R., and S. J. Galli, (1990). Mast cells as a source of both preformed and immunologically inducible TNF-Vcachectin. Nature 346:274-6.

164. Kulbe, H., Chakravarty, P., Leinster, D. A., Charles, K. A., Kwong, J., and R. G. Thompson, et al., (2012). A dynamic inflammatory cytokine network in the human ovarian cancer microenvironment. Cancer Res 72:66-75.

165. Kulbe, H., Thompson, R., Wilson, J. L., Robinson, S., Hagemann, T., and R. Fatah, et al., (2007). The inflammatory cytokine tumor necrosis factor-alpha generates an autocrine tumor-promoting network in epithelial ovarian cancer cells. Cancer Res 67:585-92.

166. Liu, F., Ai, F., Tian, L., Liu, S., Zhao, L., and X. Wang, (2016). Infliximab enhances the therapeutic effects of 5-fluorouracil resulting in tumor regression in colon cancer. Onco Targets Ther 9:5999-6008.

167. Szomolay, B., Eubank, T. D., Roberts, R. D., Marsh, C. B., and A. Friedman, (2012). Modeling the inhibition of breast cancer growth by GM-CSF. J Theor Biol 303:141-51.

168. Cheng, Y. C., Valero, V., Davis, M. L., Green, M. C., Gonzalez-Angulo, A. M., Theriault, R L, et al. (2010). Addition of GM-CSF to trastuzumab stabilises disease in trastuzumab-resistant HER2+ metastatic breast cancer patients. Br J Cancer 103:1331-4.

169. Eubank, T. D., and C. B. Marsh, (2009). GM-CSF Inhibits Breast Cancer Growth and Metastasis by Invoking an Anti-angiogenic Program in Tumor-Educated Macrophages. Cancer Res 69:2133.

170. Clifton, G. T., Gall, V., Peoples, G. E., and E. A. Mittendorf, (2016). Clinical Development of the E75 Vaccine in Breast Cancer. Breast Care (Basel) 11:116-21.

171. Mestas J, Hughes C C. Of mice and not men: differences between mouse and human immunology. J Immunol 2004; 172:2731-8.

172. Dellinger A, Zhou Z, Lenk R, MacFarland D, Kepley C L. Fullerene nanomaterials inhibit phorbol myristate acetate-induced inflammation. Exp Dermatol 2009; 18:1079-81.

All references, articles, publications, patents, patent publications, and patent applications cited herein are incorporated by reference in their entireties for all purposes. In the event that any inconsistency exists between the disclosure of the present application and the disclosure(s) of any document incorporated herein by reference, the disclosure of the present application shall govern. However, mention of any reference, article, publication, patent, patent publication, and patent application cited herein is not, and should not be taken as an acknowledgment or any form of suggestion that they constitute valid prior art. It is to be understood that, while the disclosure has been described in conjunction with the detailed description, thereof, the foregoing description is intended to illustrate and not limit the scope. Other aspects, advantages, and modifications are within the scope of the claims set forth below.

What is claimed:

1. Human mast cells obtained from adipose derived stem cells and sensitized with an immunoglobulin-E targeted to a cancer antigen.

2. A composition comprising human mast cells obtained from adipose derived stem cells and sensitized with an immunoglobulin-E targeted to a cancer antigen.

3. A method of treating a tumor in a human, comprising administering to a human a therapeutically effective amount of human mast cells obtained from adipose derived stem cells, wherein the mast cells are autologous to the human and sensitized with an immunoglobulin-E targeted to a cancer antigen.

4. The method of claim 3, wherein the human mast cells are administered in a pharmaceutically acceptable carrier.

5. The method of claim 3, wherein the administering step is subcutaneous administration, intraperitoneal administration, intratumoral administration, or intravenous administration.

6. The method of claim 3, wherein the cancer antigen is an epithelial cancer antigen, a prostate specific cancer antigen, a prostate specific membrane antigen, a bladder cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, or a colorectal cancer antigen.

7. The method of claim 6, wherein the epithelial cancer antigen is a breast cancer antigen, gastrointestinal cancer antigen, or a lung cancer antigen.

8. The method of claim 3, wherein cancer cells are killed, tumor angiogenesis is inhibited, tumor metastasis is inhibited, tumor growth is decreased, or tumor growth is inhibited.

9. A method of preparing human mast cells in vitro comprising the steps of:
obtaining human mast cells from adipose derived stem cells, and sensitizing the human mast cells with an immunoglobulin-E targeted to a cancer antigen.
